(19) United States
(12) Patent Application Publication
(10) Pub. No.: US 2023/0201366 A1
(43) Pub. Date: Jun. 29, 2023

(54) ANTI-PSMA CONJUGATES

(71) Applicants: ADC THERAPEUTICS SA, Epalinges (CH); MEDIMMUNE LIMITED, Cambridge (GB)

(72) Inventors: Patricius Hendrikus Cornelis VAN BERKEL, London (GB); Ian KIRBY, London (GB)

(21) Appl. No.: 18/056,080

(22) Filed: Nov. 16, 2022

(30) Foreign Application Priority Data
Nov. 19, 2021 (EP) 21209418.9

(51) Int. Cl. A61K 47/68; C07K 16/30; A61P 35/00

(52) U.S. Cl. CPC A61K 47/6869; A61K 47/6803; C07K 16/3069; A61P 35/00

(57) ABSTRACT

This disclosure relates to antibody conjugates comprising antibodies that bind specifically to prostate-specific membrane antigen (PSMA), conjugated to cytotoxic warheads, and associated uses.

### REFERENCE TO SEQUENCE LISTING SUBMITTED ELECTRONICALLY

The contents of the electronic sequence listing (0233-0027US1_SL.xml; Size: 11 KB; and Date of Creation Oct. 30, 2022) submitted herewith, is herein incorporated by reference in its entirety.

### FIELD OF THE INVENTION

The present disclosure relates to anti-PSMA drug conjugates and their use in therapy.

### BACKGROUND

PSMA is present on the cell surface of some normal prostatic epithelial cells, normal renal proximal tubular cells, proximal small bowel and some astrocytes (found in the brain). PSMA is highly upregulated/overexpressed on prostate cancer (Pea) cells. Expression levels of PSMA increase along with prostate cancer progression and PSMA levels in early stage prostate cancer predict a higher likelihood of recurrence. Furthermore, many solid tumours express PSMA in their tumour neovasculature whereas normal vascular endothelium is PSMA-negative.

Prostate cancer is one of the most common causes of cancer deaths in American males. In 2007, approximately 219,000 new cases are expected to be diagnosed as well as 27,000 deaths due to this disease. There is currently very limited treatment for prostate cancer once it has metastasized (spread beyond the prostate). Systemic therapy is limited to various forms of androgen (male hormone) deprivation. While most patients will demonstrate initial clinical improvement, virtually inevitably, androgen-independent cells develop. Endocrine therapy is thus palliative, not curative. Median overall survival in these patients where androgen-independent cells have developed was 28-52 months from the onset of hormonal treatment. Subsequent to developing androgen-independence, only taxane-based (i.e., docetaxel) chemotherapy has been shown to provide a survival benefit, with a median survival of 19 months. Once patients fail to respond to docetaxel, median survival is 12 months.

Where prostate cancer is localized and the patient's life expectancy is 10 years or more, radical prostatectomy offers the best chance for eradication of the disease. Historically, the drawback of this procedure is that many cancers had spread beyond the bounds of the operation by the time they were detected. However, the use of prostate-specific antigen testing has permitted early detection of prostate cancer. As a result, surgery is less extensive with fewer complications. Patients with bulky, high-grade tumours are less likely to be successfully treated by radical prostatectomy. Radiation therapy has also been widely used as an alternative to radical prostatectomy. Patients generally treated by radiation therapy are those who are older and less healthy and those with higher-grade, more clinically advanced tumours. However, after surgery or radiation therapy, if there are detectable serum prostate-specific antigen concentrations, persistent cancer is indicated. In many cases, prostate-specific antigen concentrations can be reduced by radiation treatment. However, this concentration often increases again within two years.

It has been recently shown that small molecule PSMA enzyme inhibitors could slow the growth rate of PSMA-expressing Pea cells in vitro. However, not only have these inhibitors in the past failed to have any meaningful effect on tumour cell growth in animal models, but since they act on PSMA folate hydrolase activity they have had a negative impact on whole body folate metabolism which is critical for normal physiological processes.

Accordingly, there is a need for an effective non-surgical approach to the treatment of prostate cancer and other diseases.

### SUMMARY

One approach to developing treatments for prostate cancer is the use of antibody drug conjugates that target PSMA.

ADCT-401 is an antibody drug conjugate with an anti-PSMA antibody (J591) conjugated to a pyrrolobenzodiazepine cytotoxin via a cathepsin-cleavable linker and cysteine residues on the antibody. Phase 1A clinical trials with ADCT-401 in metastatic prostate cancer showed evidence of efficacy but two issues were observed. (1) rapid pharmacokinetics of the active ADC resulting in low exposure to the ADC. (2) instability of H-L chain complex after several days, further reducing the exposure to active ADC.

We have now found that anti-PSMA antibody 2A10 can show superior stability compared to J591 and other anti-PSMA antibodies whilst the resulting PBD conjugates retain high levels of cytotoxicity.

Accordingly in a first aspect the present disclosure provides an antibody drug conjugate comprising an antibody that binds to prostate-specific membrane antigen (PSMA), conjugated to a DNA-binding cytotoxin comprising a pyrrolobenzodiazepine (PBD), wherein the antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8.

In one embodiment the antibody comprises an immunoglobulin heavy chain variable region having the amino acid sequence shown in SEQ ID NO: 1; and/or an immunoglobulin light chain variable region having the amino acid sequence shown in SEQ ID NO: 2.

In one embodiment the cytotoxin comprises a PBD dimer, for example the conjugate comprises formula (I):

Ab-L-Dp (I)

wherein:

Ab is the antibody that binds to PSMA;

L-Dp is of formula (II)

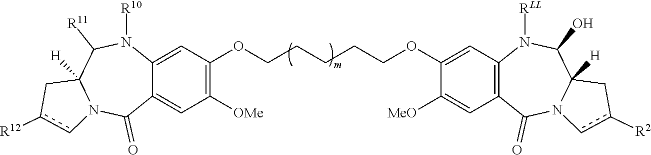

wherein:

(a) R^{LL} is a linker for connection to Ab;

(b) (i) R¹⁰ and R¹¹ together form a double bond between the nitrogen and carbon atoms to which they are attached; or (ii) R¹⁰ is R^{LLA} which is a linker for connection to Ab, and R¹¹ is OH, where R^{LL} and R^{LLA} may be the same or different; or (iii) R¹⁰ is a capping group R^{C} and R¹¹ is OH;

(c) m is 0 or 1;

(d) when there is a double bond between C2 and C3, R² is methyl;

when there is a single bond between C2 and C3, R² is either H or

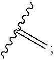

when there is a double bond between C2′ and C3′, R¹² is methyl;

when there is a single bond between C2′ and C3′, R¹² is H or

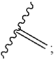

and

(e) p is the number of drug moieties (D) per Ab, such as from 1 to 8.

In one embodiment R^{LL} is of formula (IIa):

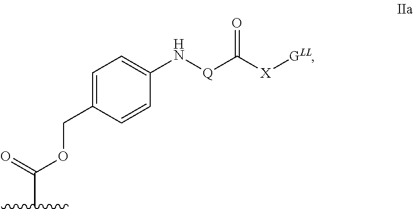

wherein:

(i) Q is:

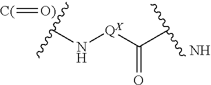

where Q^{X} is such that Q is an amino-acid residue, a dipeptide residue, a tripeptide residue or a tetrapeptide residue;

X is:

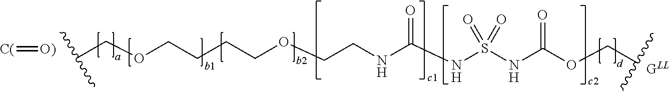

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0);

(ii) G^{LL} is a linker group connected to Ab.

The drug loading is represented by p, the number of drug units per antibody. Drug loading may range from an average of 1 to 8 Drug units (D) per antibody.

In a second aspect is provided the conjugate of the first aspect together with one or more pharmaceutically acceptable carriers or diluents, such as a pharmaceutical composition in liquid or lyophilized form. The composition may comprise a therapeutically effective amount of a chemotherapeutic agent.

The conjugate or compositions comprising it may be used in therapy. For example, in a method of treating a proliferative disease, the method comprising admistering an effective amount of the conjugate or the composition to an individual in need of such treatment.

The treated proliferative disease may be cancer, such as prostate cancer, hepatocellular carcinoma, bladder cancer, breast cancer, colorectal cancer, gastric cancer, glioblastoma, lung cancer, lymphoma, melanoma, neuroendocrine cancer, ovarian cancer, pancreatic cancer, renal cancer, squamous cell carcinoma, or sarcoma. The proliferative disease may be characterised by the presence of a neoplasm comprising both PSMA+ve and PSMA−ve cells, and/or may be a solid tumour. The proliferative disease may be characterised by the over-expression of PSMA, either in all or most of the aberrant cells, or in at least part of the tumour neovasculature.

### BRIEF DESCRIPTION OF THE FIGURES

FIG. 1 is a graph showing a pharmacokinetic assessment of the half-life of PSMA antibodies in a humanized FcRn model.

FIG. 2 is a graph showing in vivo anti-tumour activity of different antibody-conjugates in the CWR22Rv1 xenograft model.

### DETAILED DESCRIPTION

Antibody Drug Conjugates

The present disclosure provides, inter alia, an antibody drug conjugate comprising (i) a cell binding agent which comprises the complementarity determining regions of the 2A10 antibody such that the cell binding agent binds to PSMA on the surface of cell; (ii) a cytotoxin comprising a pyrrolobenzodiazapine moiety typically a PBD dimer. The cytotoxin is connected to the cell binding agent via one or more linkers—examples of suitable linkers are described in more detail below. In one embodiment each linker is connected through the N10 position on one of the PBD moieties conjugated to an antibody as defined below.

Various aspects and embodiments of the disclosures herein are thus suitable for use in providing a PBD compound to a preferred site in a subject. The conjugate in some embodiments preferably allows the release of an active PBD compound that does not retain any part of the linker.

Before transport or delivery into a cell, the antibody-drug conjugate (ADC) is preferably stable and remains intact, i.e. the antibody remains linked to the drug moiety. The linkers in such preferred embodiments are stable outside the target cell and may be cleaved at some efficacious rate inside the cell. An effective linker in some embodiments will: (i) maintain the specific binding properties of the antibody; (ii) allow intracellular delivery of the conjugate or drug moiety; (iii) remain stable and intact, i.e. not cleaved, until the conjugate has been delivered or transported to its targetted site; and (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Cell Binding Agents/Antibodies

The cell binding agent in some embodiments is an antibody that binds to PSMA and which comprises the complementarity determining regions (CDRs) of monoclonal antibody 2A10.

The term “antibody” herein is used in the broadest sense and specifically covers monoclonal antibodies, including both intact antibodies and antibody fragments, so long as they exhibit the desired biological activity, for example, the ability to bind PSMA. Antibodies may be murine, rat, human, humanized, chimeric, or derived from other species. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule.

A variety of immunoglobulin variant formats are known in the art which are derived from conventional immunoglobulins, such as bispecific antibodies, scFvs, nanobodies and the like. These are all within the scope of the term “antibody” provided they retain the 2A10 CDRs and PSMA binding activity.

Thus in some embodiments the antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8.

The CDR sequences as disclosed herein have been identified and defined using the Kabat numbering scheme (Kabat et al., U.S. Department of Health and Human Services, 1991).

In one embodiment the antibody comprises a VH domain having the sequence according to SEQ ID NO. 1. In another embodiment the antibody comprises a VL domain having the sequence according to SEQ ID NO. 2. Thus the antibody may comprise a VH domain and a VL domain where the VH comprises the sequence of SEQ ID NO. 1 and the VL domain comprises the sequence of SEQ ID NO. 2.

The VH and VL domain(s) in various embodiments form an antibody antigen binding site that binds PSMA.

In some embodiments the antibody is an intact antibody comprising a VH domain and a VL domain, the VH and VL domains having sequences of SEQ ID NO. 1 paired with SEQ ID NO. 2.

In some embodiments the antibody is the 2A10 antibody disclosed in WO2006/089230.

As used herein, “binds PSMA” is used to mean that the cell binding agent or the antibody binds PSMA with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds PSMA with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The cell binding agents or antibodies of the disclosure can in some embodiments bind PSMA with a high affinity. For example, in some embodiments the antibody can bind PSMA with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

As used herein, PSMA refers to Prostate-Specific Membrane Antigen. In one embodiment, PSMA polypeptide corresponds to Genbank accession no. AAA60209, version no. AAA60209.1 GI:190664, record update date: Jun. 23, 2010 08:48 AM. In one embodiment, the nucleic acid encoding PSMA polypeptide corresponds to Genbank accession no. M99487, version no. M99487.1 GI:190663, record update date: Jun. 23, 2010 08:48 AM.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts.

Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method or may be made by recombinant DNA methods. The monoclonal antibodies may also be isolated from phage antibody libraries or from transgenic mice carrying a fully human immunoglobulin system.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; and down regulation of cell surface receptors such as B cell receptor and BCR.

Modification of Antibodies

The antibodies disclosed herein may be modified. For example, to make them less immunogenic to a human subject. This may be achieved using any of a number of techniques familiar to the person skilled in the art. Such techniques includes humanisation to reduce the in vivo immunogenicity of a non-human antibody or antibody fragment. There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

Other sequence modification can be made to assist with conjugation of drugs or other substances of interest to particular sites in the antibody. For example one or more cysteine residues, such as in the hinge region, may be substituted or introduced, where conjugation to a cysteine residue is desired. In one embodiment the heavy chain has a cysteine residue introduced at position 242 of SEQ ID NO: 2.

PBD Cytotoxin

Pyrrolobenzodiazepines (PBDs) suitable for use in the present disclosure have the ability to recognise and bond to specific sequences of DNA; the preferred sequence is PuGPu. PBDs are of the general structure:

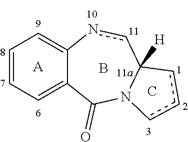

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

The cytotoxin is typically a PBD dimer, such as a PBD dimer, which together with the linker(s) and any optional capping group, has the following formula (II):

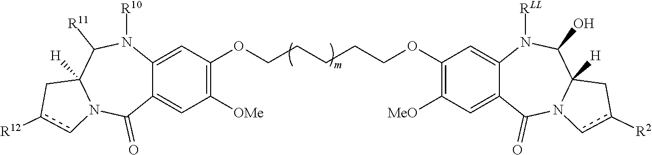

wherein R^{LL} is a linker for connection to the antibody, with examples and particular embodiments provided in more detail below in the section entitled ‘Linkers’.

When released from the linker R^{LL}, the bond between N10, to which R^{LL} was originally attached, and C11 typically together form a double bond between the nitrogen and carbon atoms to which they are attached (an imine (C═N)). Similarly, in the released drug, R¹⁰ and R¹¹ typically together form a double bond between the nitrogen and carbon atoms to which they are attached. Thus the released drug may be of formula RelA:

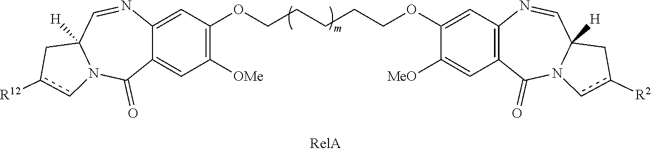

R¹⁰ and R¹¹

In some embodiments, R¹⁰ and R¹¹ together form a double bond between the nitrogen and carbon atoms to which they are attached.

In another embodiment, R¹⁰ is a linker R^{LLA} for connection to Ab and R¹¹ is OH, with R^{LLA} having the same definition as R^{LL}, and R^{LLA} and R^{LL} being the same or different.

In another embodiment, R¹⁰ is a capping group R^{C} and R¹¹ is OH.

A capping group can be used to reduce or inhibit the cytotoxic activity of the PBD, effectively forming a prodrug, but in this context is not linked to the cell binding agent. The capping group is then removed, for example in the target cell or in the tumour microenviroment, to activate the drug. Various capping groups are described in Franzyk and Christiansen, 2021, Molecules 26: 1292. (1) Prodrugs cleaved in acidic media e.g. salts of dithiocarbamates. (2) Prodrugs cleaved by reactive oxygen species. (3) Prodrugs cleaved by glutathione. (4) Prodrugs cleaved by expressed enzymes, such as oxidoreductases, hydrolases and matrix metalloproteinases. (5) Prodrugs cleaved by beta-glucuronidase, e.g. R^{C} may comprise a glucuronide, for example:

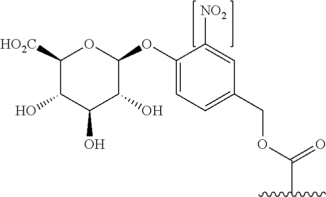

Wherein the square brackets indicate the NO₂ group is optional. In one embodiment, the NO₂ group is present.

A capping group may also be used to modify the physicochemical characteristics of the antibodody drug conjugate e.g. to make it more stable. For example, the capping group may increase the hydrophilicity of the antibody drug conjugate.

m

In some embodiments, m is 0. In some embodiments, m is 1.

R² and R¹²

In some embodiments, R² and R¹² are the same.

In some embodiments, there is a double bond between C2 and C3 and between C2′ and C3, and R² and R¹² are both methyl.

In some embodiments, there is a single bond between C2 and C3 and between C2′ and C3, and R² and R¹² are both H.

In some embodiments, there is a single bond between C2 and C3 and between C2′ and C3, and R² and R¹² are both

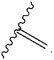

In a preferred embodiment, the drug-linker, L-D is of formula (III)

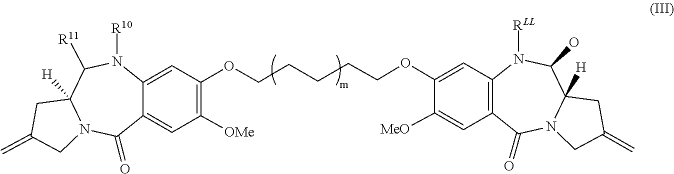

Where R^{LL}, R¹⁰, R¹¹ and m are as defined above:

In a particular embodiment, R¹⁰, and R¹¹ together form a double bond between the nitrogen and carbon atoms to which they are attached; and m is 0 (i.e. SG2000 with a linker at N10).

In another embodiment, the drug-linker, L-D is of formula (IV):

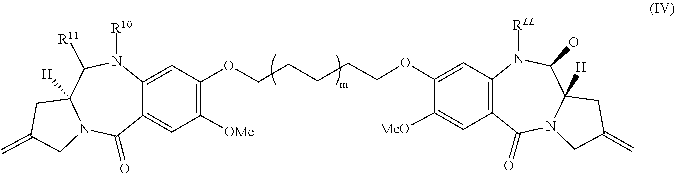

where R^{LL}, R¹⁰, R¹¹ and m are as defined above.

In a particular embodiment, R¹⁰, and R¹¹ together form a double bond between the nitrogen and carbon atoms to which they are attached; and m is 1 (i.e. SG3199 with a linker at N10).

In another embodiment, L-D is of formula V (of which formula II is an example):

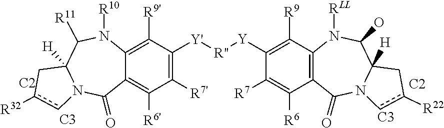

Where R^{LL}, R¹⁰ and R¹¹ are as defined above.

R²² and R³²

In one embodiment, when there is a double bond present between C2 and C3, R²² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

(id)

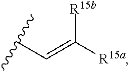

wherein each of R⁴¹, R⁴² and R⁴³ are independently selected from H, C₁₋₃ saturated alkyl, C₂₋₃ alkenyl, C₂₋₃ alkynyl and cyclopropyl, where the total number of carbon atoms in the R¹² group is no more than 5;

(ie)

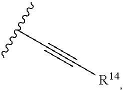

wherein one of R^{15a} and R^{15b} is H and the other is selected from: phenyl, which phenyl is optionally substituted by a group selected from halo, methyl, methoxy; pyridyl; and thiophenyl; and

(if)

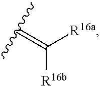

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl.

In another embodiment when there is a single bond present between C2 and C₃,

R²² is

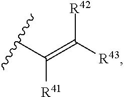

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester.

In one embodiment, when there is a double bond present between C2′ and C3′, R³² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

(id)

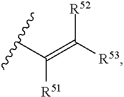

wherein each of R⁵¹, R⁵² and R⁵³ are independently selected from H, C₁₋₃ saturated alkyl, C₂₋₃ alkenyl, C₂₋₃ alkynyl and cyclopropyl, where the total number of carbon atoms in the R¹² group is no more than 5;

(ie)

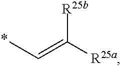

wherein one of R^{25a} and R^{25b} is H and the other is selected from: phenyl, which phenyl is optionally substituted by a group selected from halo, methyl, methoxy; pyridyl; and thiophenyl; and

(if)

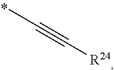

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl.

In another embodiment when there is a single bond present between C2′ and C3′,

R¹² is

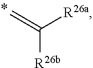

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester.

Particular embodiments of R²² and R³² are set out in formulae II, III IV.

R⁶, R⁷, R⁹, R″, Y and Y′

R⁶ and R⁹ are independently selected from H, R, OH, OR, SH, SR, NH₂, NHR, NRR′, nitro, Me₃Sn and halo; where R and R′ are independently selected from optionally substituted C₁₋₁₂ alkyl, C₃₋₂₀ heterocyclyl and C₆₋₂₀ aryl groups;

R⁷ is selected from H, R, OH, OR, SH, SR, NH₂, NHR, NHRR′, nitro, Me₃Sn and halo.

In one embodiment R⁶ and R⁹ are both H.

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH.

R^{6′}, R^{7′}, R^{9′} are selected from the same groups as R⁶, R⁷ and R⁹ respectively.

Typically when released inside the cell the resulting PBD has a C═N imine bond formed between N10 and C11, as shown below:

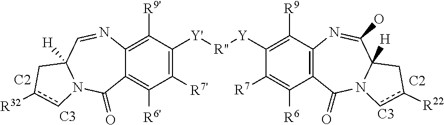

This is the active substance that binds to DNA.

Drug-Linkers

A wide variety of linker technologies are available in the art to link cytotoxins to cell binding agents. Linkers can incorporate various different moieties to assist with antibody-drug conjugate stability and determine drug release characteristics. For example the linker may include a cleavable moiety, such as one that is cleavable by cathepsin B (e.g. Valine-Alanine or Valine-Citrulline). Another strategy is to use a pH-sensitive linker whereby the lower pH of the endosome and lysosome compartments the hydrolysis of an acid-labile group within the linker, such as a hydrazone. Alternative a linker may be non-cleavable, which can avoid or reduce off-target effects and improve plasma stability during circulation.

The functionality that allows conjugation to the cell binding agent is based on the site of conjugation and its chemistry. N-hydroxysuccinimide esters are a common choice for functionalizing amines, especially when coupling to ϵ-lysine residues. For conjugation to cysteines, thiol-reactive maleimide is the most applied reactive handle, although it is also possible to create a disulfide bridge by oxidation with a linker bearing a sulfhydryl group. Aldehyde or keto functional groups such as oxidized sugar groups or pAcPhe unnatural amino acids can be reacted with hydrazides and alkoxyamines to yield acid-labile hydrazones or oxime bonds. In addition, a hydrazine can be coupled with an aldehyde via HIPS ligation to generate a stable C—C linkage.

More recent approaches have been based on the N-linked glycosylation site in antibodies, such as Asn-297 in IgG molecules. GlycoConnect™ (Synaffix), using enzymes to trim the N-linked glycans to a GlcNAc core and then a further enzymatic process to introduce an activated moiety comprising azide which can then be used to incorporate the drug-linker using copper-free click chemistry.

Other aspects of linker chemistry include spacers and/or moieties which mask the hydrophobicity of the cytotoxin payload, reduce cellular efflux mechanisms and/or increase overall stability, such as a polyethylene glycol (PEG) chain within the linker or a polar functional group such as a sulphonyl.

In some embodiments, the antibody drug conjugates of the disclosure can be described as Ab-L-D, where Ab is the anti-PSMA antibody, D is the PBD-containing cytotoxin and L is a linker. The number of Drug moieties per Ab (the drug loading, p) depends on the number of linkers attached to each Ab, and the number of Drug moieties per linker. Typically the drug loading, p, is from 1 to 8, such as from 1 to 4, 1 to 2, or 2 to 4. Where site-specific approaches are used, such as the N-linked glycosylation site at Asn-297 or where mutations have been made to reduce the number of endogenous cysteine sites available for conjugation, the number of sites available for conjugation may be limited to two per antibody. If only one drug molecule is present in L-D then p will be a maximum of 2. In some embodiments one Drug moiety is joined to each linker whereas in others, more than one Drug moiety may be joined to each linker (e.g. a branched linker). Drug loading is typically considered on an average basis since variations can arise from the conjugation process (a composition comprising a plurality of antibody drug conjugate molecules will typically have individual molecules with from zero to the maximum number of drug molecules possible). Methods for determining average drug loading are known in the art.

In one embodiment the linker (e.g. shown as R^{LL} in formula (II), (III) and (IV)) is of formula (IIa):

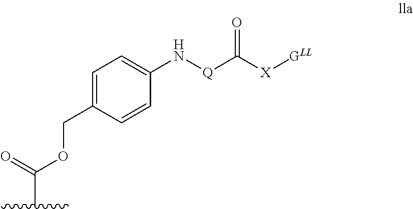

wherein

Q is:

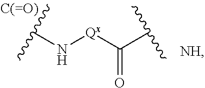

where Q^{X} is such that Q is an amino-acid residue, a dipeptide residue, a tripeptide residue or a tetrapeptide residue;

X (as connected to G^{LL}) is:

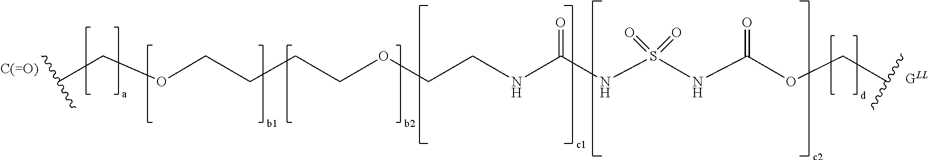

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0); and

a may be 0, 1, 2, 3, 4 or 5. In some embodiments, a is 0 to 3. In some of these embodiments, a is 0 or 1. In further embodiments, a is 0. In further embodiments, a is 1.

b1 may be 0, 1, 2, 3, 4, 5, 6, 7, 8, 9, 10, 11, 12, 13, 14, 15 or 16. In some embodiments, b1 is 0 to 12. In some of these embodiments, b1 is 0 to 8, such as from 2 to 8, and may be 0, 2, 3, 4, 5 or 8. In further embodiments, b1 is 2 or 8.

b2 may be 0, 1, 2, 3, 4, 5, 6, 7, 8, 9, 10, 11, 12, 13, 14, 15 or 16. In some embodiments, b2 is 0 to 12. In some of these embodiments, b2 is 0 to 8, such as from 2 to 8, and may be 0, 2, 3, 4, 5 or 8. In further embodiments, b2 is 2 or 8.

Only one of b1 and b2 may not be 0.

c1 may be 0 or 1.

c2 may be 0 or 1.

Only one of c1 and c2 may not be 0.

d may be 0, 1, 2, 3, 4 or 5. In some embodiments, d is 0 to 3. In some of these embodiments, d is 1 or 2. In further embodiments, d is 2. In further embodiments, d is 5.

In some embodiments of X, a is 0, b1 is 0, c1 is 1, c2 is 0 and d is 2, and b2 may be from 0 to 8. In some of these embodiments, b2 is 0, 2, 3, 4, 5 or 8. In further embodiments, b2 is 8.

In some embodiments of X, a is 1, b2 is 0, c1 is 0, c2 is 1, d is 2, and b1 may be from 0 to 8. In some of these embodiments, b1 is 0, 2, 3, 4, 5 or 8. In further embodiments, b1 is 2.

G^{LL} is a linker group connected to Ab (as defined further below);

Q^{X}

In one embodiment, Q is an amino acid residue. The amino acid may be a natural amino acid or a non-natural amino acid.

In one embodiment, Q is selected from: Phe, Lys, Val, Ala, Cit, Leu, Ile, Arg, and Trp, where Cit is citrulline.

In one embodiment, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q is selected from:

^{NH}-Phe-Lys-^{C=O},
^{NH}-Val-Ala-^{C=O},
^{NH}-Val-Lys-^{C=O},
^{NH}-Ala-Lys-^{C=O},
^{NH}-Val-Cit-^{C=O},
^{NH}-Phe-Cit-^{C=O},
^{NH}-Leu-Cit-^{C=O},
^{NH}-Ile-Cit-^{C=O},
^{NH}-Phe-Arg-^{C=O},
^{NH}-Trp-Cit-^{C=O}
and
^{NH}-Gly-Val-^{C=O};

where Cit is citrulline.

Preferably, Q is selected from:

^{NH}-Phe-Lys-^{C=O},
^{NH}-Val-Ala-^{C=O},
^{NH}-Val-Lys-^{C=O},
^{NH}-Ala-Lys-^{C=O},
and
^{NH}-Val-Cit-^{C=O}

Most preferably, Q is selected from ^{NH}-Phe-Lys-^{C═O}, ^{NH}-Val-Cit-^{C═O} or ^{NH}-Val-Ala-^{C═O}.

Other dipeptide combinations of interest include:

^{NH}-Gly-Gly-^{C=O},
^{NH}-Gly-Val-^{C=O}
^{NH}-Pro-Pro-^{C=O},
and
^{NH}-Val-Glu-^{C=O}

Other dipeptide combinations may be used, including those described by Dubowchik et al., Bioconjugate Chemistry, 2002, 13, 855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:

^{NH}-Glu-Val-Ala-^{C=O}
^{NH}-Glu-Val-Cit-^{C=O}
^{NH}-αGlu-Val-Ala-^{C=O}
^{NH}-αGlu-Val-Cit-^{C=O}

In some embodiments, Q is a tetrapeptide residue. The amino acids in the tetrapeptide may be any combination of natural amino acids and non-natural amino acids. In some embodiments, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:

(SEQ ID NO: 10)
^{NH}-Gly-Gly-Phe-Gly^{C=O};
and
(SEQ ID NO: 11)
^{NH}-Gly-Phe-Gly-Gly^{C=O}.

In some embodiments, the tetrapeptide is:

^{NH}-Gly-Gly-Phe-Gly^{C=O},

In the above representations of peptide residues, NH— represents the N-terminus, and —C═O represents the C-terminus of the residue. The C-terminus binds to the NH attached to the benzene ring.

Glu represents the residue of glutamic acid, i.e.:

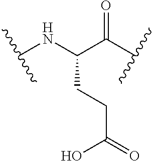 [SMILES: *N[C@@H](CCC(=O)O)C(*)=O]

αGlu represents the residue of glutamic acid when bound via the α-chain, i.e.:

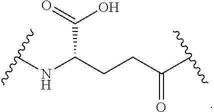 [SMILES: *N[C@@H](CCC(*)=O)C(=O)O]

In one embodiment, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

G^{LL}

G^{LL} may be selected from:

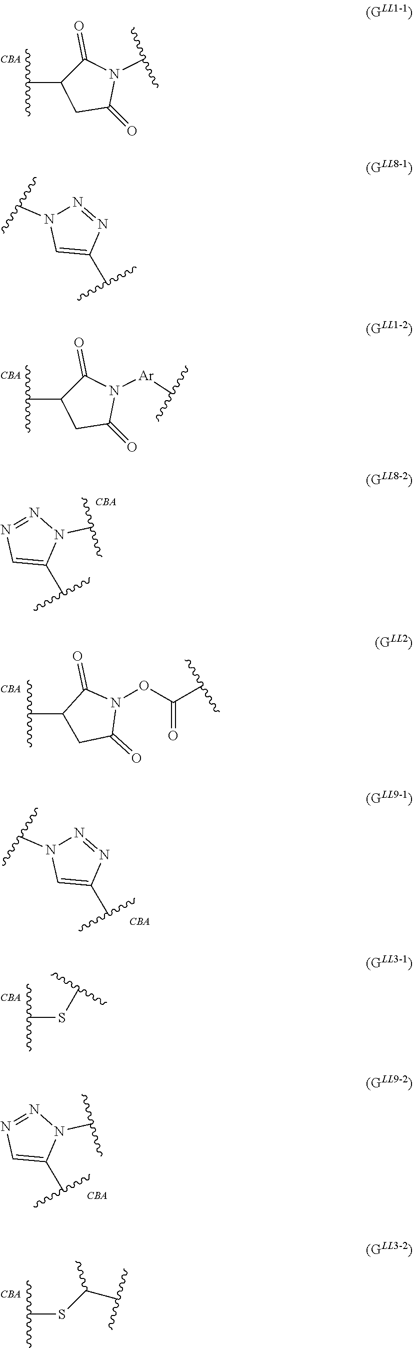

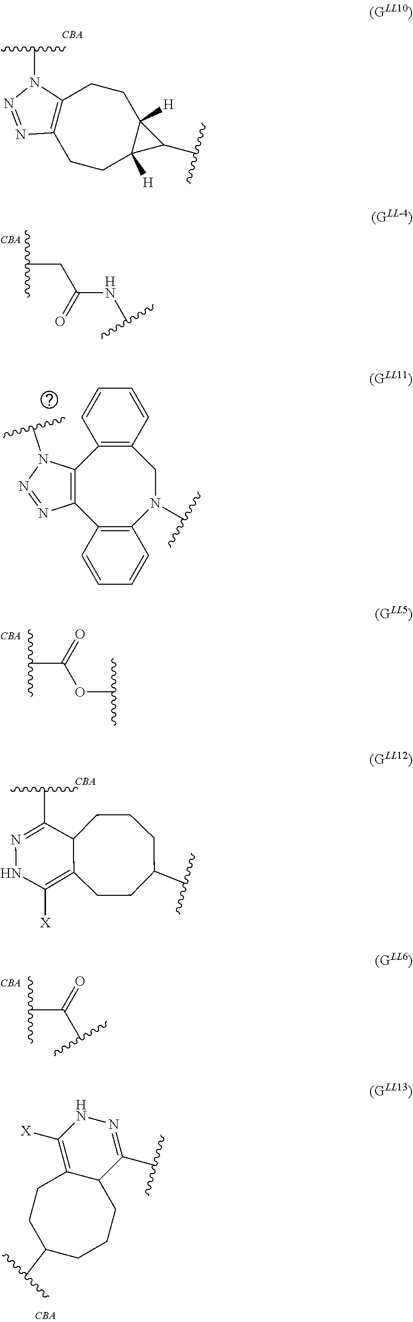

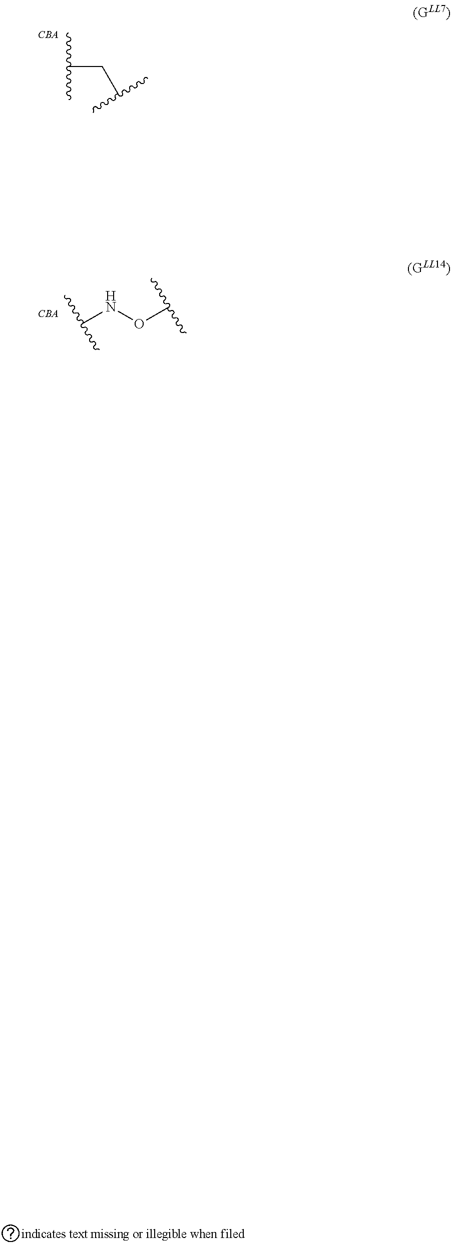

where Ar represents a C₅₋₆ arylene group, e.g. phenylene and X represents C₁₋₄ alkyl. CBA indicates the end of G^{LL} connected to the antibody.

In some embodiments, G^{LL} is selected from G^{LL1-1} and G^{LL1-2}. In some of these embodiments, G^{LL} is G^{LL1-1}.

In other embodiments, G^{LL} is selected from G^{LL10} and G^{LL11}. In some of these embodiments, G^{LL} is G^{LL10}.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and

N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

The linker is typically connected to the PBD dimer via the N10 position, such as is shown in the location of R^{LL} in the example embodiments below.

Drug Linker-Embodiments

In some embodiments, the drug-linker (L-D) is selected from:

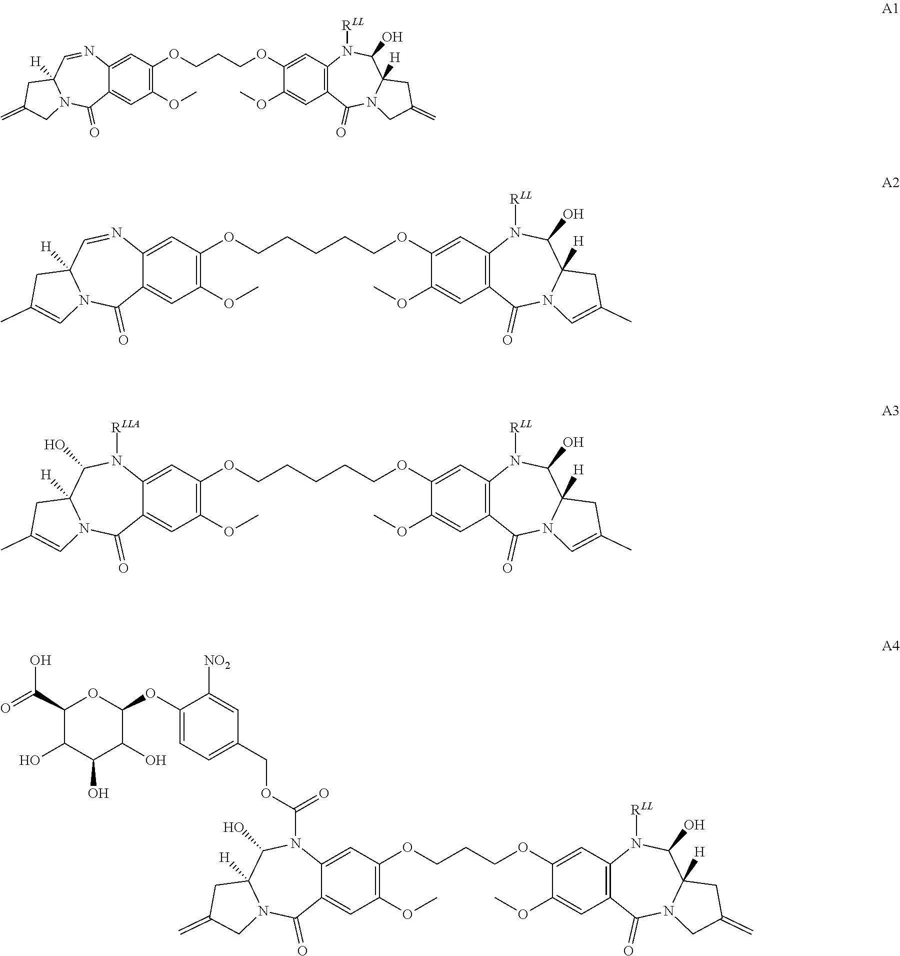

where R^{LL} and R^{LLA} are as described above.

In some embodiments, L-D is selected from:

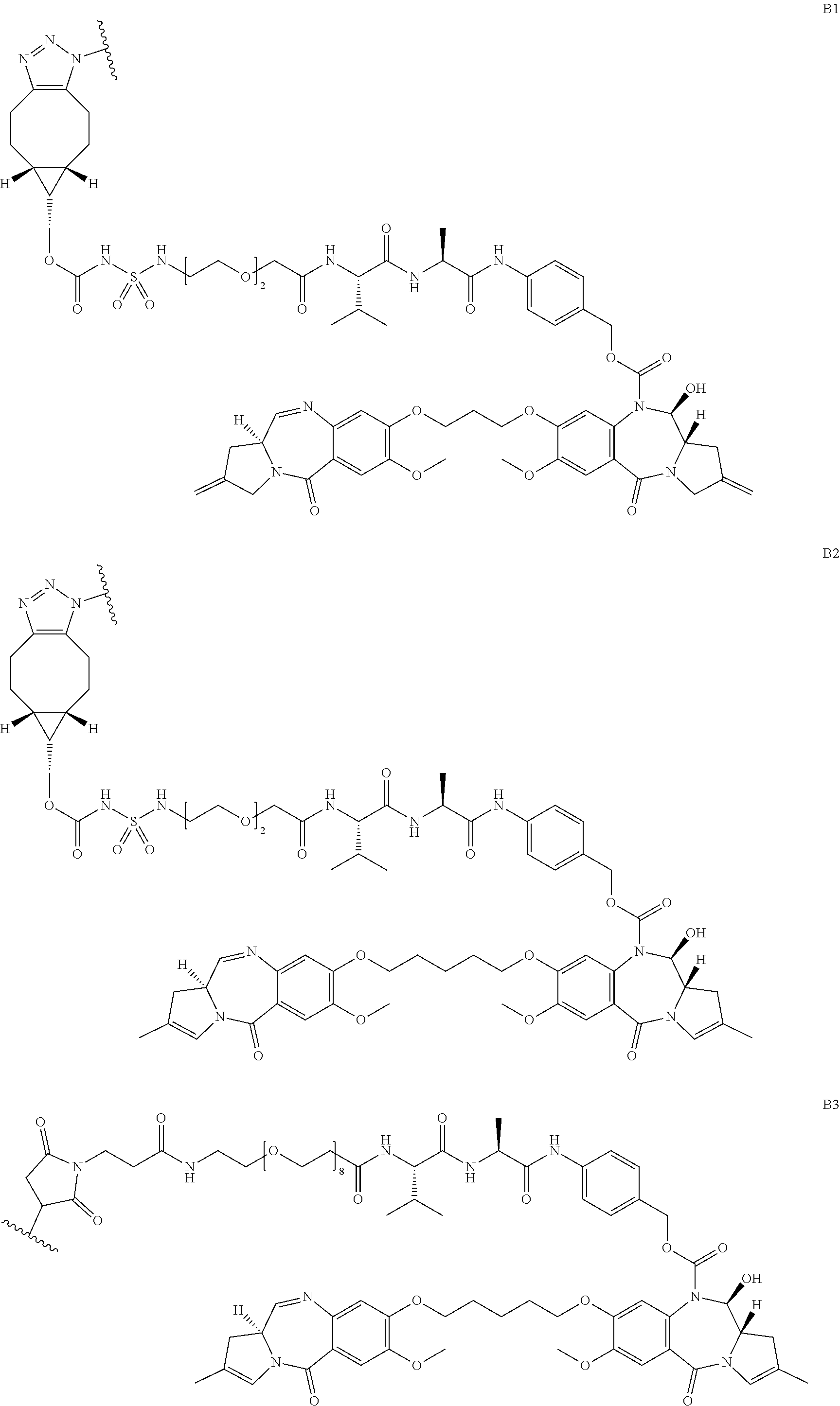

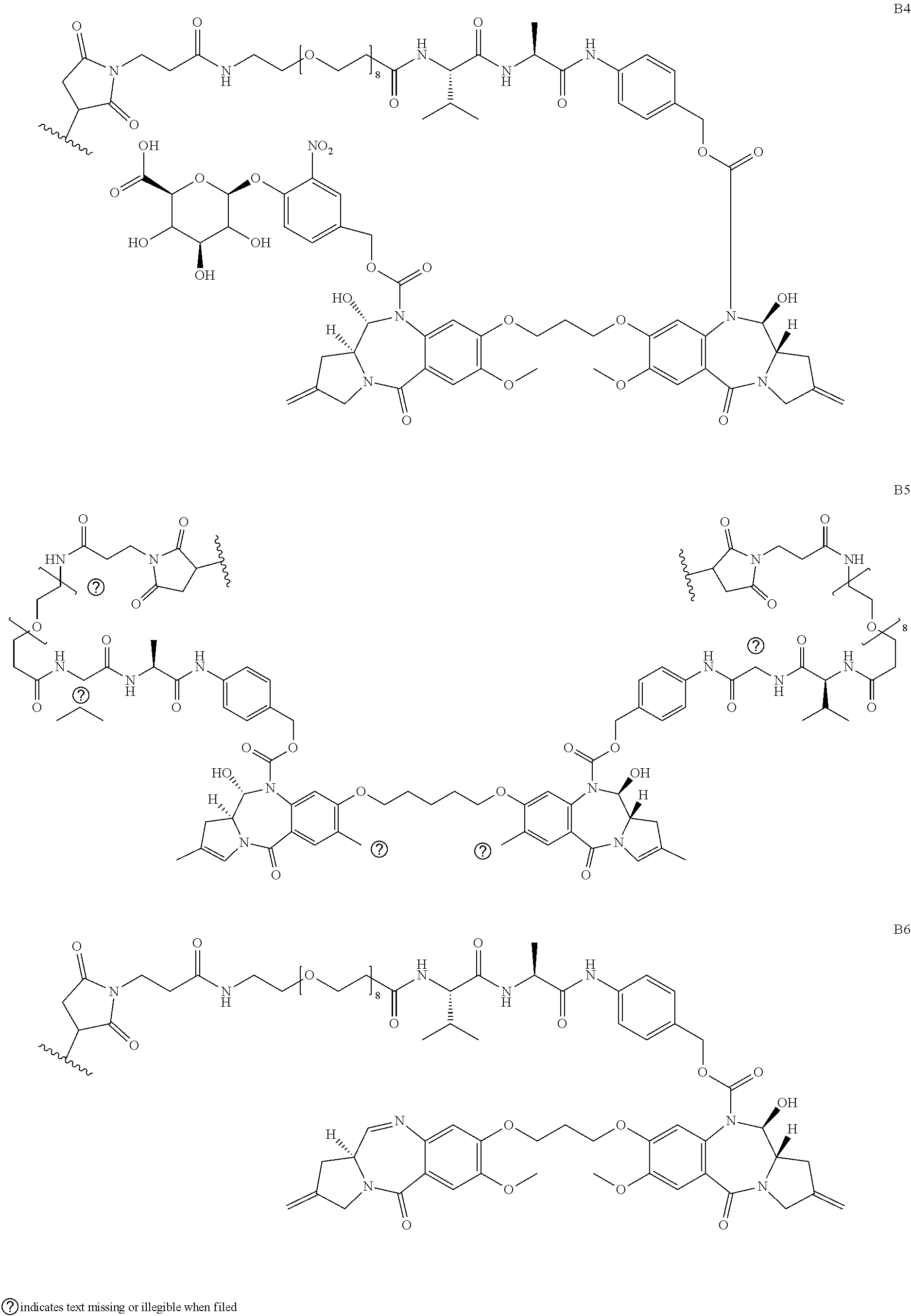

Site of Conjugation and Drug Loading

Drug-linkers can be conjugated to a cell binding agent, such as an antibody, using a variety of methods known in the art and at a number of different sites. Conjugation sites include cysteine residues and lysine residues in the antibody sequence (endogenous or engineered), as well as sites of N-linked glycosylation following trimming (e.g. the GlycoConnect™ or GlyClick™ approaches). Thus in one embodiment the drug-linker is conjugated via a trimmed Asn-GlcNAc residue, typically at the endogenous N-linked glycosylation site in the antibody (Asn-297). The GlcNAc residue may be linked to a derivatized sugar residue, such as GalNAc (e.g. as results from the use of the GlycoConnect™ approach). The derivatized sugar may include a reactive group, such as an azide, which can react with a complementary reactive group on the remainder of the linker, such as dibenzocyclooctyne (DBCO) or BCN (bicyclononyne)—see C1/C2 and B1/B2 as examples. With respect to cysteine conjugation, in one embodiment the cysteine is an endogenous cysteine located in the hinge region or Fc domain. In another embodiment the cysteine in an engineered cysteine introduced in the hinge region or Fc domain.

The drug loading is the average number of PBD drugs per cell binding agent, e.g. antibody, in a composition comprising a plurality of molecules.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10: 7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the drug-linker intermediate or linker reagent. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody, and which do not form intrachain or intermolecular disulfide linkages (Junutula, et al., 2008b Nature Biotech., 26(8):925-932; Dornan et al (2009) Blood 114(13):2721-2729; U.S. Pat. Nos. 7,521,541; 7,723,485; WO2009/052249). The engineered cysteine thiols may react with linker reagents or the drug-linker reagents of the present disclosure which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody.

In some embodiments, the reactive group on the antibody may be modified to be present, for example azide. In this case, p is limited by the number of attachment sites on the antibody, i.e. the number of azide groups. For example, the antibody may have only one or two azide groups to which the drug linker may be attached.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per antibody is in the range 1 to 8. In some embodiments the range is selected from 1 to 4, 1 to 2, 2 to 4, and 1 to 3.

In some embodiments, there are one or two dimer pyrrolobenzodiazepine groups per antibody.

Where L-D has two linking groups, these are preferably to the same antibody. In some of these embodiments, only one L-D is attached to each antibody, so the drug loading is 1 or an average of from 0.7 to 1.

Preparation of Drug Conjugates

The antibody drug conjugates of the present disclosure may be prepared by conjugating the drug-linker, such as the following drug linker (of formula (II)—as previously defined herein) to the antibody:

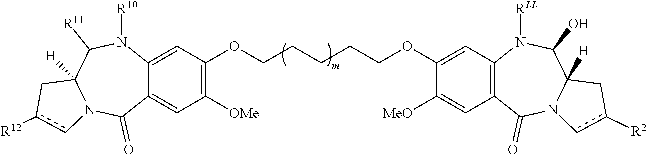

As described above, a number of conjugation techniques are known in the art, such as (i) conjugation to an endogenous or engineered cysteine residue via maleimide, as for example described in U.S. Pat. No. 9,889,207 or Flynn et al., 2016. Mol Cancer Ther 15: 2709—as would be applicable to compounds C₃, C4, C5 and C6 below; and (ii) using GlyClick or GlycoConnect to attached via chemoenzymatically-trimmed N-linked glycosylation site, e.g. at Asn-297 or its equivalent, as described in WO2018/146188 which describes the use of EndoS to trim glycan isoforms to core GlcNAc, followed by enzymatic transfer to the core GlcNAc of a N-acetylgalactose residue harboring an azide group for conjugation to the drug linker, typically using Galactose Transferase (GalT) or Galactose-N-acetyl Transferase (GalNAcT) enzyme. If a GalT enzyme is used, preferably the enzyme incorporates the Y289L and/or the C342T mutations. Finally, the drug-linker is reacted with the azide group using copper-free click chemistry, such as the method described in van Geel, R., et al., Bioconjugate Chemistry, 2015, 26, 2233-2242. This method would be applicable to compounds C1 and C2 below.

The drug linker may be synthesised as described in, for example, Tibergien et al., 2016, ACS Med. Chem. Lett. 7: 983-987, WO2014/057074, WO2018/069490 and WO2018/146188.

In particular, the following table provides references for each of the drug linkers of particular interest.

| | | |
|---|---|---|
| C1 | See compound 4 below | 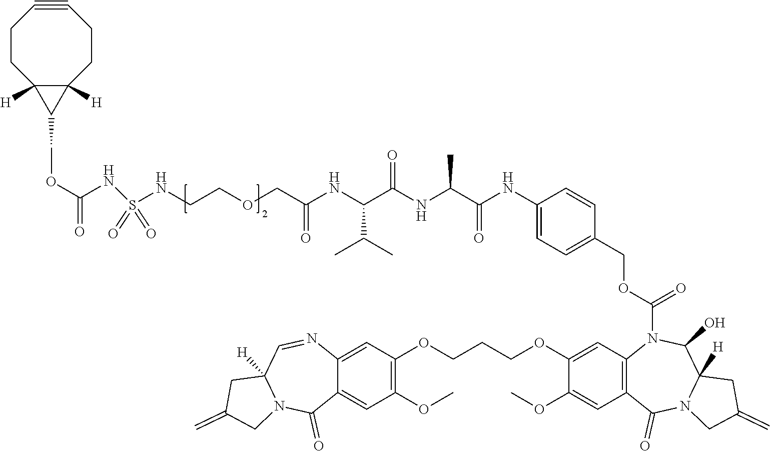 |
| C2 | WO2018/146188 Compound 4 | 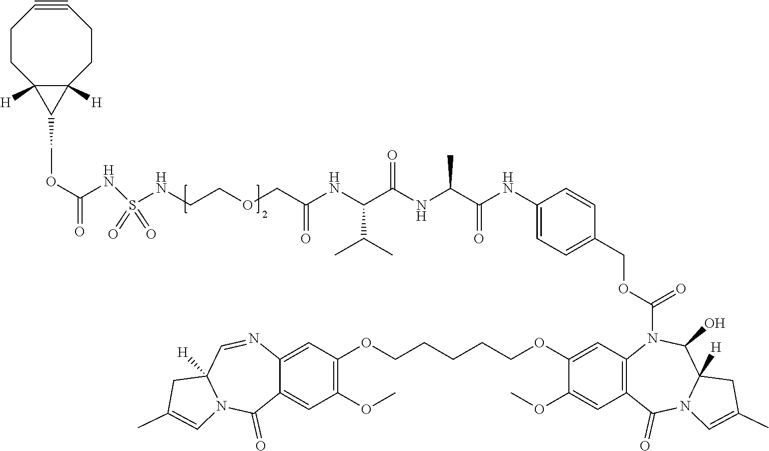 |
| C3 | WO2014/057074 Compound 24 | 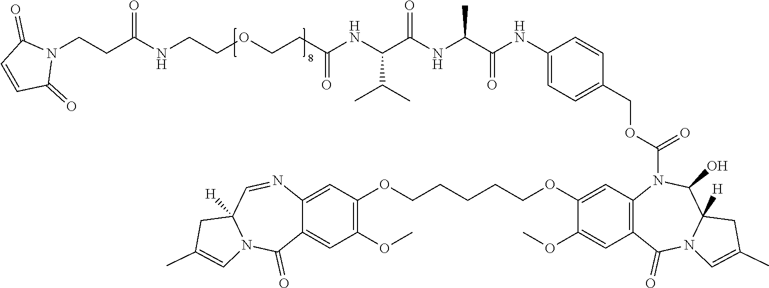 |
| C4 | WO2018/069490 Compound 10 | 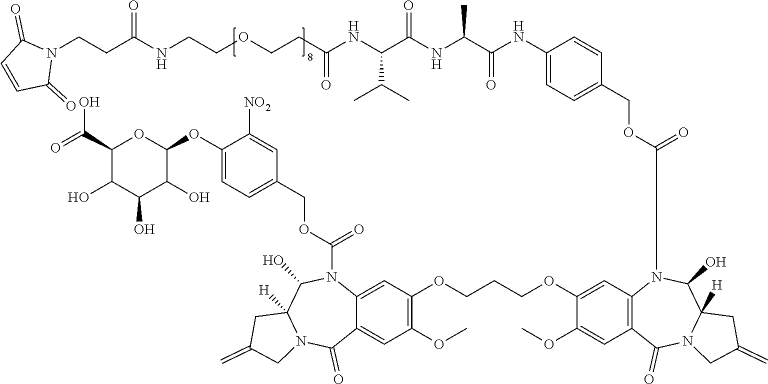 |
| C5 | WO2019/034764 Compound 10 | 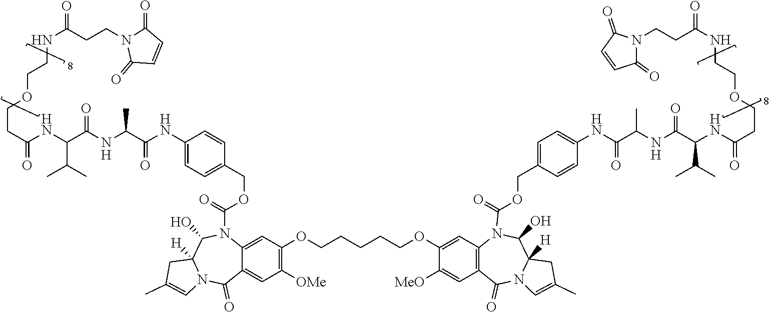 |
| C6 | WO2017/137553 Compound 23 | 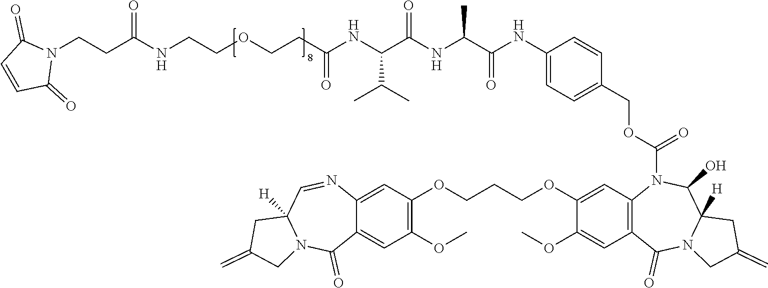 |

Synthesis of C1

Analytical LC/MS Conditions

Positive mode electrospray mass spectrometry was performed using a Waters Aquity H-class SQD2. Mobile phases used were solvent A (water with 0.1% formic acid) and solvent B (acetonitrile with 0.1% formic acid).

Method 1: Gradient for routine 3-minute run: Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Detection was at 254 nm. Column: Waters Acquity UPLC™ BEH Shield RP18 1.7 μm 2.1×50 mm at 50° C. fitted with Waters Acquity UPLC™ BEH Shield RP18 VanGuard Pre-column, 130A, 1.7 μm, 2.1 mm×5 mm.

Method 2: Gradient for 15-minute run: Initial composition 5% B held over 1 minute, then increased from 5% B to 100% B over a 9 minute period. The composition was held for 2 minutes at 100% B, then returned to 5% B in 10 seconds and held there for 2 minutes 50 seconds. The total duration of the gradient run was 15.0 minutes. Flow rate was 0.8 mL/minute (for 3-minute run) and 0.6 mL/minute (for 15-minute run). Detection was at 254 nm. Column: ACE Excel 2 C18-AR, 2μ, 3.0×100 mm fitted with Waters Acquity UPLC™ BEH Shield RP18 VanGuard Pre-column, 130A, 1.7 μm, 2.1 mm×5 mm.

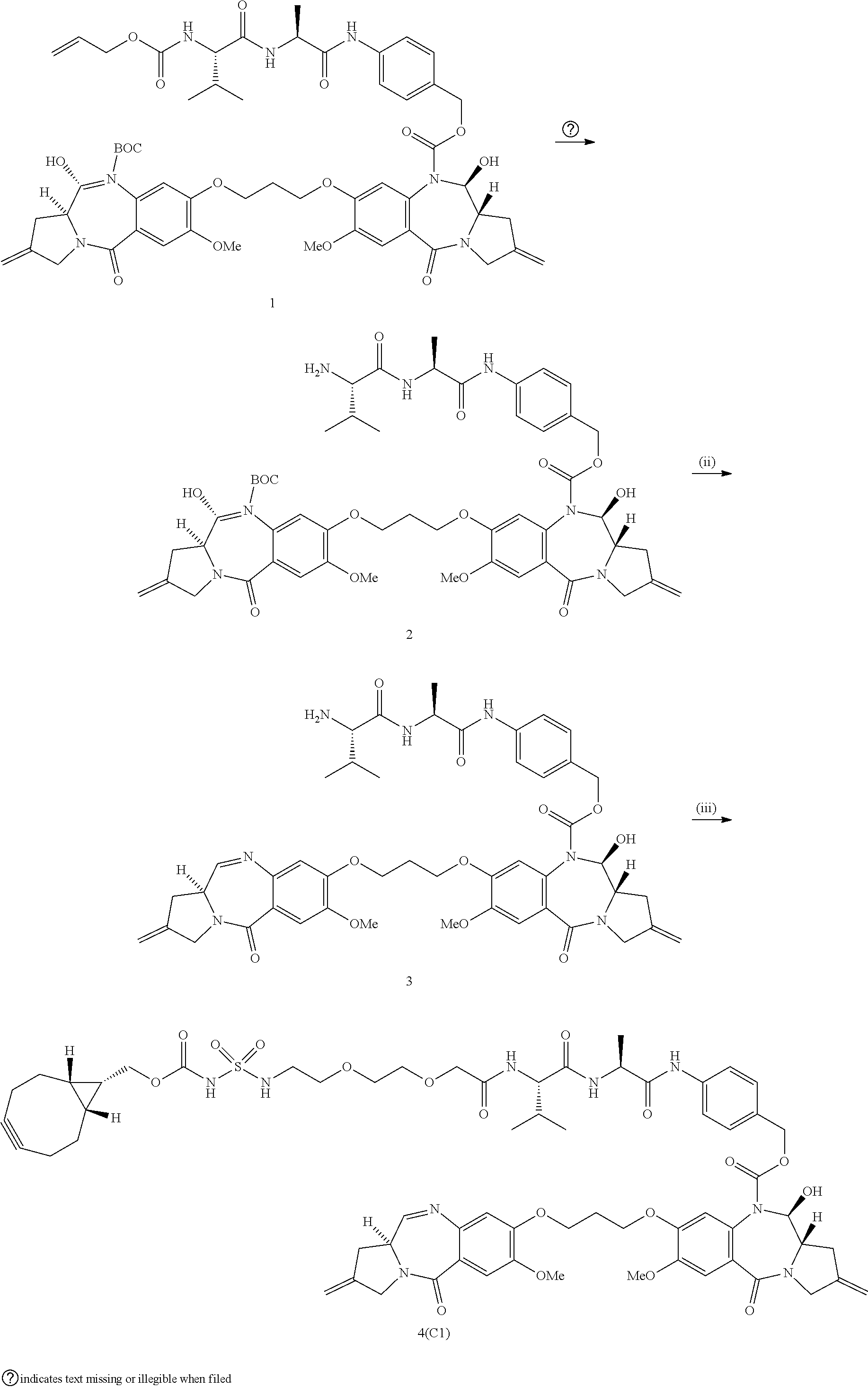

(i) Alloc Deprotection

Tetrakis(triphenylphosphine)palladium(0) (8.4 mg, 2 mol %) was added to a solution of (1) (400 mg, 0.36 mmol, 1.0 eq)(Compound 21 in WO2017/137553) and pyrrolidine (38 μL, 0.46 mmol, 1.25 eq) in chloroform (10 mL). The reaction was stirred for 20 minutes at room temperature, LCMS shows complete reaction. The reaction mixture was diluted with chloroform (5 mL), washed with saturated aqueous ammonium chloride (10 mL) and passed through a phase separator tube to remove traces of water. Deloxan™ (1 g) was added to the organic phase and stirred at room temperature for 60 mins. The Deloxan was removed by filtration, washed with chloroform (5 mL) and the organic fractions evaporated under reduced pressure to leave 2 as a white solid (335 mg, 94%). LC/MS, method 1, 1.30 min (ES+) m/z 1013.1 ([M+H]⁺).

(ii) Boc Deprotection

A mixture of TFA (4.5 mL) and water (0.5 mL) was cooled to 0° C. and added to 2 (320 mg, 0.31 mmol). The resulting solution was stirred at 0° C. for 2 hr. Water (5 mL) and chloroform (10 mL) were added and the mixture basified (pH 8) by the addition of solid sodium hydrogen carbonate. The organic phase was removed by passing through a phase separator cartridge, and evaporated to dryness under reduced pressure to leave 3 as an off-white solid (216 mg, 77%). LC/MS, method 1, 1.23 min (ES+) m/z 894.9 ([M+H]⁺).

(iii) BCN Hydraspace™ Coupling

EDCI·HCl (86 mg, 0.45 mmol, 1.1 eq) was added to a solution of 3 (200 mg, 0.22 mmol, 1.0 eq) and BCN spacer (108 mg, 0.26 mmol, 1.15 eq)(compound 3 in WO2018/146188) in chloroform (10 mL) and the resulting reaction stirred at room temperature for 60 min. LCMS showed no starting material to be present. The organic phase washed with water (10 mL). The resulting mixture was passed through a phase separator to remove the water and evaporated to dryness to leave a yellow solid which was purified by prep HPLC (gradient 30-90% acetonitrile/water over 9 min. The water containing 0.01% formic acid, but no acid in the acetonitrile). The crude material was dissolved in acetonitrile (1.3 mL) and water (0.7 mL) and injected in 100 μL batches. The product was collected in tubes containing 5% aqueous ammonium bicarbonate solution (2 mL). The fractions containing product were combined, the acetonitrile removed under reduced pressure and the resulting aqueous phase extracted with DCM (3×50 mL). The organic fractions were dried by passing through a phase separator and evaporated to dryness to leave the product as a pale yellow solid (75 mg, 26%). LC/MS, method 2, 6.78 min (ES+) m/z 1295.3 ([M+H]⁺).

Treated Disorders

The therapies described herein include those with utility for anticancer activity. In particular, in certain aspects the therapies include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides therapies comprising administering a conjugate compound as described herein, which binds to PSMA, for use in therapy, wherein the method comprises selecting a subject based on expression of PSMA protein.

In one aspect, the present disclosure provides a therapy with a label that specifies that the therapy is suitable for use with a subject determined to be suitable for such use. The label may specify that the therapy is suitable for use in a subject has expression of PSMA e.g. is a PSMA+ve cancer. The label may specify that the subject has a particular type of cancer.

The label may specify that the subject has a PSMA+ve cancer.

The range of disorders that may be treated by such therapies is described in more detail below.

In a further aspect there is also provided a therapy as described herein for use in the treatment of a proliferative disease. Another aspect of the present disclosure provides the use of a conjugate compound as described herein in the manufacture of a medicament for treating a proliferative disease.

One of ordinary skill in the art is readily able to determine whether or not a candidate therapy treats a proliferative condition for any particular cell type. For example, assays which may conveniently be used to assess the activity offered by a particular compound are described below.

The therapies described herein may be used to treat a proliferative disease. The term “proliferative disease” pertains to an unwanted or uncontrolled cellular proliferation of excessive or abnormal cells which is undesired, such as, neoplastic or hyperplastic growth, whether in vitro or in vivo.

The proliferative disease may be characterised by the presence of a neoplasm comprising both PSMA+ve and PSMA−ve cells.

The target neoplasm or neoplastic cells may be all or part of a solid tumour, such as an advanced solid tumour.

Thus in one embodiment the neoplasm/cancer is itself essentially PSMA+ve. This includes prostate cancer.

In addition, previous studies have found that PSMA is upregulated on the endothelial cells of the neovasculature of a wide variety of solid tumors where it may facilitate endothelial cell sprouting and invasion through its regulation of lytic proteases that have the ability to cleave the extracellular matrix (Van de Wiele et al., 2020, Histopathol. 35(9): 919:927). Accordingly, conjugate compounds described herein may be used to treat solid tumours by targeting PSMA expressed in the neovasculature of such tumours.

Thus examples of proliferative conditions that may be treated with the conjugate compounds described herein include, but are not limited to, benign, pre-malignant, and malignant cellular proliferation, including but not limited to, neoplasms, tumours and cancers, such as histocytoma, glioma, glioblastoma, astrocyoma, osteoma, lung cancer, small cell lung cancer, non-small cell lung cancer, gastrointestinal cancer, gastric cancer, bowel cancer, colon cancer, colorectal cancer, hepatocellular carcinoma, breast cancer, ovarian cancer, prostate cancer, testicular cancer, liver cancer, kidney/renal cancer, bladder cancer, pancreatic cancer, brain cancer, head and neck cancer, thyroid cancer, neuroblastoma, neuroendocrine cancer, sarcoma, osteosarcoma, Kaposi's sarcoma, squamous cell carcinoma, melanoma, and lymphomas.

In a particular embodiment, the condition is selected from renal cell cancer, bladder transitional cell carcinoma, colonic adenocarcinoma, hepatocellular carcinoma, neuroendocrine carcinoma, glioblastoma, melanoma, pancreatic cancer, such as pancreatic duct carcinoma, soft tissue sarcoma, ovarian cancer, endometrial cancer, breast cancer, colorectal, gastric and lung cancer such as non-small cell lung carcinoma, mesothelioma. PSMA is positive in the neovasculature of these tumours.

Prostate cancer, adenoid cystic carcinoma of the head and neck, and thyroid cancer are cancers of particular interest.

Patient Selection

In certain aspects, the individuals are selected as suitable for treatment with the treatments before the treatments are administered.

As used herein, individuals who are considered suitable for treatment are those individuals who are expected to benefit from, or respond to, the treatment. Individuals may have, or be suspected of having, or be at risk of having cancer. Individuals may have received a diagnosis of cancer. In particular, individuals may have, or be suspected of having, or be at risk of having, prostate cancer. Typically the individual is an animal or human subject.

In some aspects, individuals are selected on the basis of the amount or pattern of expression of a first target protein. In some aspects, the selection is based on expression of a first target protein at the cell surface.

In some cases, individuals are selected on the basis they have, or are suspected of having, are at risk of having cancer, or have received a diagnosis of a proliferative disease characterised by the presence of a neoplasm comprising cells having a high level of surface expression of PSMA. The neoplasm may be composed of cells having a high level of surface expression of PSMA. In some cases, high levels of surface expression means that mean number of anti-PSMA antibodies bound per neoplastic cell is greater than 70000, such as greater than 80000, greater than 90000, greater than 100000, greater than 110000, greater than 120000, greater than 130000, greater than 140000, or greater than 150000.

In some cases, individuals are selected on the basis they have, or are suspected of having, are at risk of having cancer, or have received a diagnosis of a proliferative disease characterised by the presence of a neoplasm comprising cells having a low level of surface expression of PSMA. The neoplasm may be composed of cells having a low level of surface expression of PSMA. In some cases, low levels of surface expression means that mean number of anti-PSMA antibodies bound per neoplastic cell is less than 20000, such as less than 80000, less than 70000, less than 60000, less than 50000, less than 40000, less than 30000, less than 20000, less than 10000, or less than 5000.

In some aspects, individuals are selected on the basis they have a neoplasm comprising both PSMA+ve and PSMA-ve cells. The neoplasm may be composed of PSMA-ve neoplastic cells, optionally wherein the PSMA-ve neoplastic cells are associated with PSMA+ve neoplastic or non-neoplastic cells. The neoplasm or neoplastic cells may be all or part of a solid tumour. The solid tumour may be partially or wholly PSMA-ve. In one embodiment, the expression of PSMA is found in the tumour neovasculature.

In some cases, expression of PSMA in a particular tissue of interest is determined. For example, in a sample of prostate tissue or tumor tissue. In some cases, systemic expression of the target is determined. For example, in a sample of circulating fluid such as blood, plasma, serum or lymph.

In some aspects, the individual is selected as suitable for treatment due to the presence or absence of PSMA expression in a sample. In those cases, individuals without PSMA expression may be considered not suitable for treatment.

In other aspects, the level of PSMA expression is used to select a individual as suitable for treatment. Where the level of expression of PSMA is above a threshold level, the individual is determined to be suitable for treatment.

In some aspects, the presence of PSMA in cells in the sample indicates that the individual is suitable for treatment with an ADC as disclosed herein. In other aspects, the amount of PSMA expression must be above a threshold level to indicate that the individual is suitable for treatment. In some aspects, the observation that PSMA localisation is altered in the sample as compared to a control indicates that the individual is suitable for treatment.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a first target protein. In some aspects disclosed herein, a patient is determined to be suitable for treatment if at least at least 10% of the cells in the sample express a first target protein.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a second target protein. In some aspects disclosed herein, a patient is determined to be suitable for treatment if at least at least 10% of the cells in the sample express a second target protein.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a first target polypeptide or nucleic acid in the blood, in white blood cells, peripheral blood mononuclear cells, granulocytes and/or red blood cells.

In another aspect the sample is a biopsy of solid tissue e.g. one that could include the neovasculature of a solid tumour if present.

The sample may be fresh or archival. For example, archival tissue may be from the first diagnosis of an individual, or a biopsy at a relapse. In certain aspects, the sample is a fresh biopsy.

The terms “subject”, “patient” and “individual” are used interchangeably herein.

In some aspects disclosed herein, an individual has, or is suspected as having, or has been identified as being at risk of, a proliferative disease such as cancer. In some aspects disclosed herein, the individual has already received a diagnosis of such a disease. A list of relevant diseases is provided above. Prostate cancer, adenoid cystic carcinoma of the head and neck, and thyroid cancer are cancers of particular interest.

In some cases, the individual has received a diagnosis of a proliferative disease such as cancer, such as one of the disorders listed above. Prostate cancer, adenoid cystic carcinoma of the head and neck, and thyroid cancer are cancers of particular interest.

In some cases, the individual has received a diagnosis of a solid cancer containing PSMA+ expressing cells, such as PSMA+ expressing cells in the tumour neovasculature.

The individual may be undergoing, or have undergone, a therapeutic treatment for that cancer. The subject may, or may not, have previously received an anti-PSMA ADC. In some cases the cancer is prostate cancer.

Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated.

Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the ADC alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, anti-metabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Also included in the definition of “chemotherapeutic agent” are: (i) anti-hormonal agents that act to regulate or inhibit hormone action on tumors such as anti-estrogens and selective estrogen receptor modulators (SERMs); (ii) aromatase inhibitors that inhibit the enzyme aromatase, which regulates estrogen production in the adrenal glands; (iii) anti-androgens; (iv) protein kinase inhibitors such as MEK inhibitors; (v) lipid kinase inhibitors; (vi) anti-angiogenic agents).

Also included in the definition of “chemotherapeutic agent” are therapeutic antibodies.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which will typically be by injection, e.g. cutaneous, subcutaneous, or intravenous.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Dosage

It will be appreciated by one of skill in the art that appropriate dosages of the ADC, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of PSMA observed in a sample obtained from the subject. Thus, the level or localisation of expression of PSMA in the sample may be indicative that a higher or lower dose of ADC is required. For example, a high expression level of PSMA may indicate that a higher dose of ADC would be suitable. In some cases, a high expression level of PSMA may indicate the need for administration of another agent in addition to the ADC. For example, administration of the ADC in conjunction with a chemotherapeutic agent. A high expression level of PSMA may indicate a more aggressive therapy.

In certain aspects, the dosage level is determined by the expression of PSMA on neoplastic cells in a sample obtained from the subject. For example, when the target neoplasm is composed of, or comprises, neoplastic cells expressing PSMA.

In certain aspects, the dosage level is determined by the expression of PSMA on cells associated with the target neoplasm. For example, the target neoplasm may be a solid tumour composed of, or comprising, neoplastic cells that express PSMA. For example, the target neoplasm may be a solid tumour composed of, or comprising, neoplastic cells that do not express PSMA. The cells expressing PSMA may be neoplastic or non-neoplastic cells associated with the target neoplasm.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

For the ADC, where it is a PBD bearing ADC, the dosage amounts described above may apply to the conjugate (including the PBD moiety and the linker to the antibody) or to the effective amount of PBD compound provided, for example the amount of compound that is releasable after cleavage of the linker.

### Specific Embodiments

Embodiment 1—An antibody drug conjugate comprising an antibody that binds to prostate-specific membrane antigen (PSMA), conjugated to a DNA-binding cytotoxin comprising a pyrrolobenzodiazepine (PBD), wherein the antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8.

Embodiment 2—the conjugate of embodiment 1 with the proviso that where the PBD is conjugated to the antibody via the following linker,

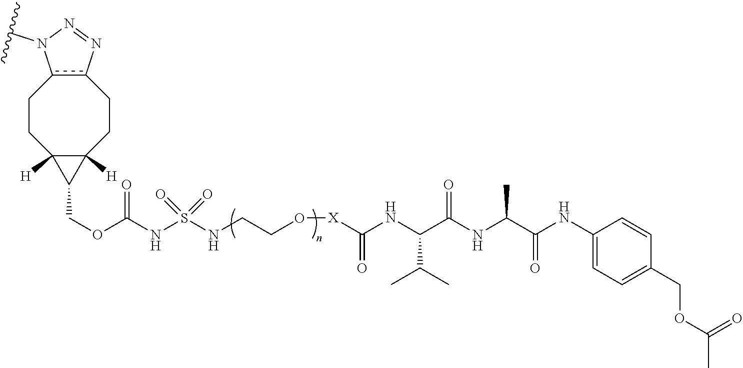

wherein X is selected from the group consisting of: a single bond, —CH₂— and —C₂H₄—; and n is from 1 to 8;
the conjugate comprises a drug linker of formula (III)

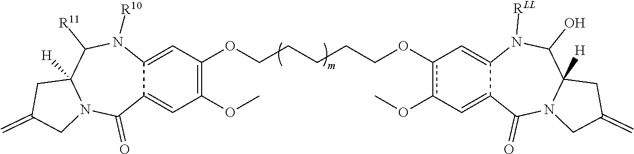

wherein:

(a) R^{LL} is a linker for connection to the antibody; and

(b) (i) R¹⁰ and R¹¹ together form a double bond between the nitrogen and carbon atoms to which they are attached; (ii) R¹⁰ is a linker R^{LLA} for connection to the antibody and R¹¹ is OH; or (iii) R¹⁰ is a capping group R^{C} and R¹¹ is OH; and

(c) m is 0 or 1.

In a particular embodiment, R¹⁰ and R¹¹ together form a double bond between the nitrogen and carbon atoms to which they are attached, and m is 0.

Embodiment 3—a conjugate of embodiment 1 or embodiment 2 where an antibody drug conjugate comprising any of the following drug linkers (A) to (G) is specifically excluded:

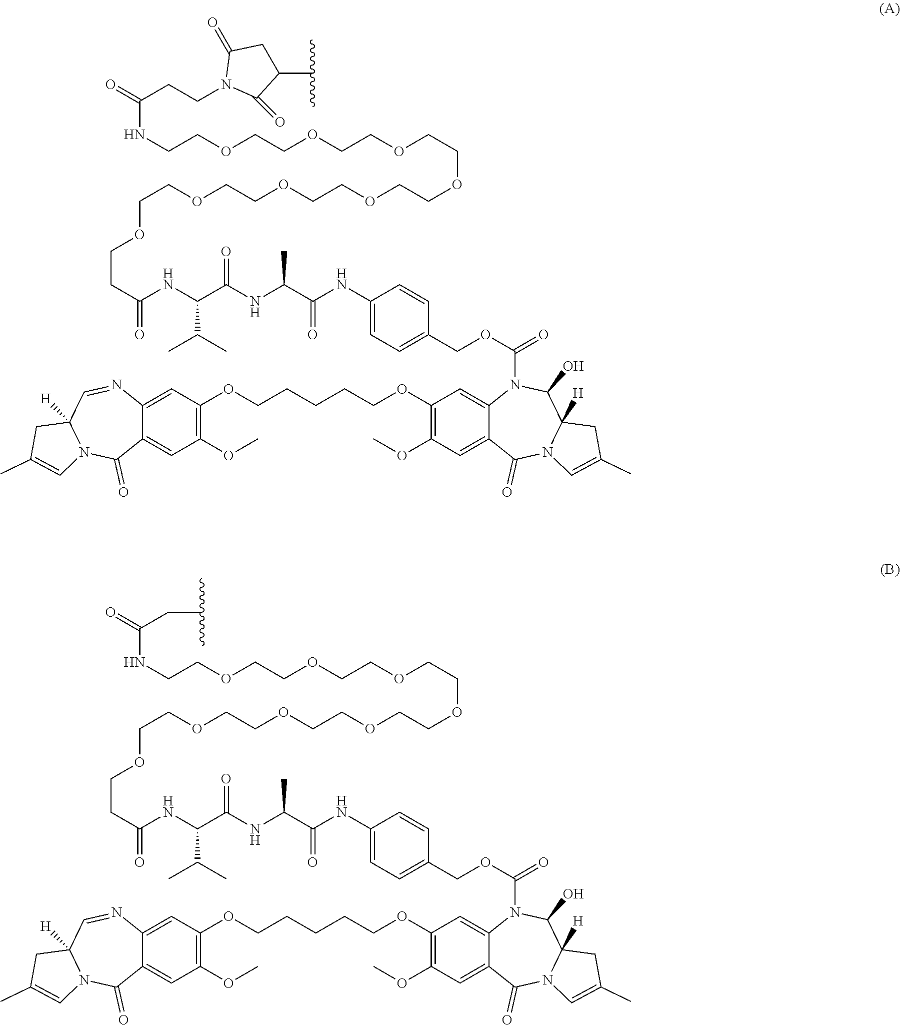

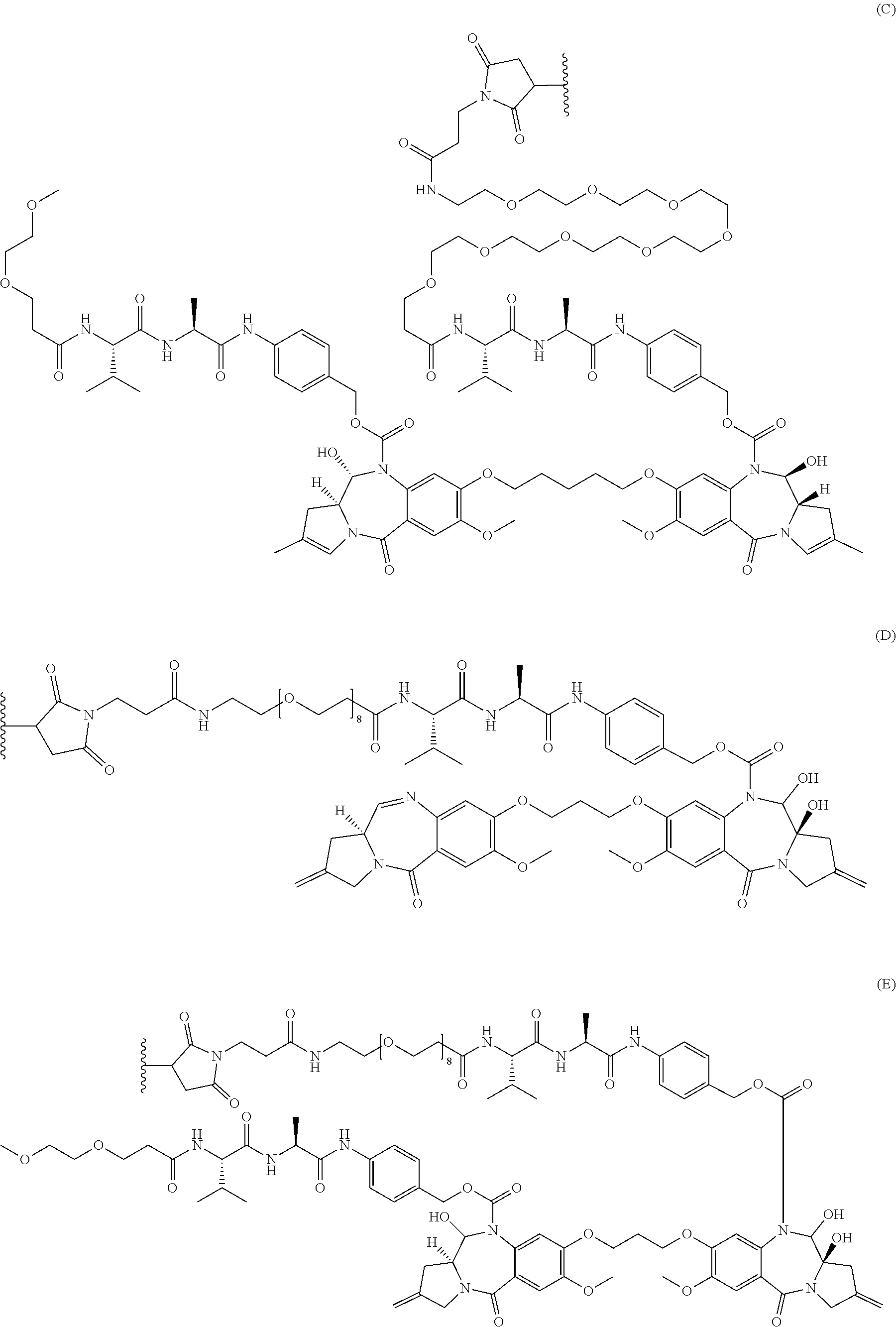

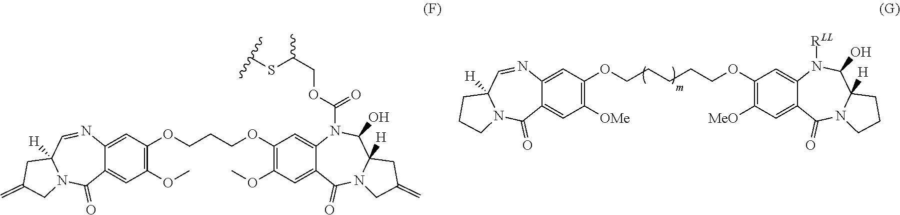

where (a) R^{LL} is a linker for connection to the antibody; and (b) m is 0 or 1.

Embodiment 4—A conjugate according to any one of embodiments 1 to 3 wherein the antibody comprises an immunoglobulin heavy chain variable region having the amino acid sequence shown in SEQ ID NO: 1.

Embodiment 5—A conjugate according to any one of the previous embodiments wherein the antibody comprises an immunoglobulin light chain variable region having the amino acid sequence shown in SEQ ID NO: 2.

Embodiment 6—A conjugate according to any one of the previous embodiments wherein the cytotoxin comprises a PBD dimer.

Embodiment 7— An antibody drug conjugate of formula (I):

Ab-L-D (I)

wherein:

Ab is the antibody as defined in embodiment 1 that binds to PSMA;

L-D is of formula (II):

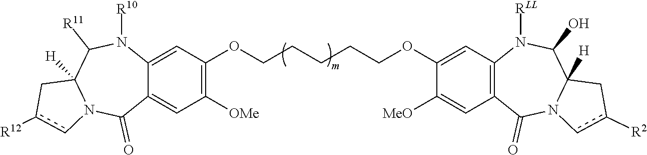

wherein:

(a) R^{LL} is a linker for connection to Ab.

(b) (i) R¹⁰ and R¹¹ together form a double bond between the nitrogen and carbon atoms to which they are attached; or (ii) R¹⁰ is R^{LLA} which is a linker for connection to Ab, and R¹¹ is OH; or (iii) R¹⁰ is a capping group R^{C} and R¹¹ is OH;

(c) m is 0 or 1; and

(d) when there is a double bond between C2 and C3, R² is methyl;

when there is a single bond between C2 and C3, R² is either H or

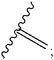

when there is a double bond between C2′ and C3′, R¹² is methyl;

when there is a single bond between C2′ and C3′, R¹² is H or

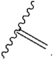

Embodiment 8—A conjugate according to embodiment 6 or 7 wherein L-D is of formula (III), as shown above, such as where the PBD dimer is SG2000.

Embodiment 9—A conjugate according to embodiment 6 or 7 wherein L-D is of formula IV, as shown above, such as where the PBD dimer is SG3199.

Embodiment 10—A conjugate according to any one of the previous embodiments wherein the conjugate comprises a linker between the cytotoxin and the antibody, which linker has the formula R^{LL} as described herein.

Embodiment 11—A conjugate according to any one of the previous embodiments wherein the conjugate comprises a cleavable inker between the cytotoxin and the antibody, such as a linker comprising a cathepsin cleavable sequence e.g. Val-Ala or Val-Cit.

Embodiment 12—A conjugate according to any one of the previous embodiments where the drug-linker is selected from compounds B1, B2, B3, B4 and B5.

Embodiment 13—A conjugate according to any one of the previous embodiments where the cytotoxin is conjugated to the antibody at an endogenous and/or engineered N-linked glycosylation site, such as Asn-297 or its equivalent.

Embodiment 14—A pharmaceutical composition comprising a conjugate according to any one of the previous embodiments together with one or more pharmaceutically acceptable carriers or diluents, such as a pharmaceutical composition in liquid or lyophilized form.

Embodiment 15—A conjugate according to any one of embodiments 1 to 13 or a pharmaceutical composition according to embodiment 14 for use in therapy, such as treating a proliferative disorder in an individual, for example a disease characterized by over-expression of PSMA.

Embodiment 16—A conjugate according to any one of embodiments 1 to 13 or a pharmaceutical composition according to embodiment 14 for use in treating prostate cancer, adenoid cystic carcinoma of the head and neck, or thyroid cancer in an individual.

Embodiment 17—A method of treating an individual suffering from a proliferative disorder, for example a disease characterized by over-expression of PSMA, which method comprises administering to the individual a conjugate according to any one of embodiments 1 to 13 or a pharmaceutical composition according to embodiment 14.

Embodiment 18—A method of treating an individual patient suffering from prostate cancer, adenoid cystic carcinoma of the head and neck, or thyroid cancer which method comprises administering to the individual a conjugate according to any one of embodiments 1 to 13 or a pharmaceutical composition according to embodiment 14.

Sequence Listing Part of the Description

| [2A10 VH] | |
|---|---|
| SEQ ID NO. 1 | |
| EVQLVQSGAE VKKPGESLKI SCKGSGYSFT SNWIGWVRQM PGKGLEWMGI IYPGDSDTRY | 60 |
| SPSFQGQVTI SADKSISTAY LQWSSLKASD TAMYYCARQT GFLWSSDLWG RGTLVTVSS | 119 |
| [2A10 VL] | |
| SEQ ID NO. 2 | |
| AIQLTQSPSS LSASVGDRVT ITCRASQDIS SALAWYQQKP GKAPKLLIYD ASSLESGVPS | 60 |
| RFSGYGSGTD FTLTINSLQP EDFATYYCQQ FNSYPLTFGG GTKVEIK | 107 |
| [2A10 VH, Kabat CDR1] | |
| SEQ ID NO. 3 | |
| SNWIG | |
| [2A10 VH, Kabat CDR2] | |
| SEQ ID NO. 4 | |
| IIYPGDSDTR YSPSFQG | |
| [2A10 VH, Kabat CDR3] | |
| SEQ ID NO. 5 | |
| QTGFLWSSDL | |
| [2A10 VL, Kabat CDR1] | |
| SEQ ID NO. 6 | |
| RASQDISSAL A | |
| [2A10 VL, Kabat CDR2] | |
| SEQ ID NO. 7 | |
| DASSLES | |
| [2A10 VL, Kabat CDR3] | |
| SEQ ID NO. 8 | |
| QQFNSYPLT | |
| [MAIA (C239i-Heaw chain] | |
| SEQ ID NO. 9 | |
| EVQLVQSGAE VKKPGESLKI SCKGSGYSFT SNWIGWVRQM PGKGLEWMGI IYPGDSDTRY | 60 |
| SPSFQGQVTI SADKSISTAY LQWSSLKASD TAMYYCARQT GFLWSSDLWG RGTLVTVSSA | 120 |
| STKGPSVFPL APSSKSTSGG TAALGCLVKD YFPEPVTVSW NSGALTSGVH TFPAVLQSSG | 180 |
| LYSLSSVVTV PSSSLGTQTY ICNVNHKPSN TKVDKKVEPK SCDKTHTCPP CPAPELLGGP | 240 |
| SCVFLFPPKP KDTLMISRTP EVTCVVVDVS HEDPEVKFNW YVDGVEVHNA KTKPREEQYN | 300 |
| STYRVVSVLT VLHQDWLNGK EYKCKVSNKA LPAPIEKTIS KAKGQPREPQ VYTLPPSRDE | 360 |
| LTKNQVSLTC LVKGFYPSDI AVEWESNGQP ENNYKTTPPV LDSDGSFFLY SKLTVDKSRW | 420 |
| QQGNVFSCSV MHEALHNHYT QKSLSLSPGK | 450 |

Some aspects and embodiments of the disclosure are described below in more detail with reference to the following examples, which are illustrative only and non-limiting.

### Example 1

ADCT-401 is an antibody drug conjugate with an anti-PSMA antibody (J591) conjugated to a pyrrolobenzodiazepine cytotoxin via a cathepsin-cleavable linker and cysteine residues on the antibody. Phase 1A clinical trials with ADCT-401 in metastatic prostate cancer showed some evidence of efficacy but two issues were observed. (1) rapid pharmacokinetics of the active ADC resulting in low exposure to the ADC. (2) instability of H-L chain complex after several days, further reducing the exposure to active ADC.

We investigated whether a different linker system (GlycoConnect™) would address these issues.

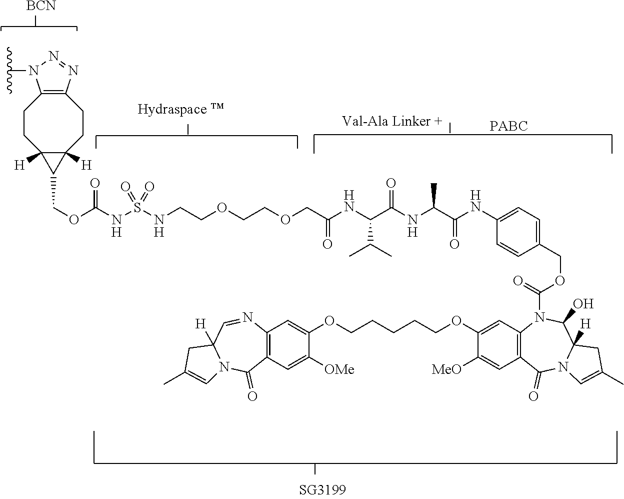

The linker is joined to the antibody at the N297 N-linked glycosylation sites following chemoenzymatic glucan trimming.

As shown in table 1, this resolved the instability of the H-L chain complex and also led to improved tolerability in rat and cynomolgus monkey, and as such improved the therapeutic index considerably.

TABLE 1

| | Efficacy | Cyno Tox | Therapeutic Index |
|---|---|---|---|
| ADCT-401 | 0.5 | 0.6 | 1 |
| J591K-PL1601 | 0.25* | >0.9 | >4 |

However, when dosed at 0.6 mg/kg in monkeys, terminal half-life did not improve significantly, although there was evidence that AUC improved significantly with a modest increase in dose (0.9 mg/kg) (data not shown).

Therefore we sought to identify an alternative anti-PSMA antibody with improved characteristics.

### Example 2— Assessment of Binding and Cytotoxicity of Different Anti-PSMA Antibodies/Drug Conjugates

We screened alternative antibodies versus J591 using ELISA, cell-based PSMA binding studies (both with naked antibodies), in vitro cytotoxicity assays and in vivo anti-tumor activity assays in the CWR22Rv1 xenograft model (both with antibodies conjugated to tesirine, the same linker-payload in ADCT-401).

TABLE 2
Anti-PSMA antibodies tested

| | |
|---|---|
| J415 | BZL Biologies Inc. (Liu et al., 1997, |
| | Cancer Res. 57: 3629-3634) |
| MDX 2A10 | U.S. Pat. No. 7,875,278 |
| PMF-A10 | WO2010/037836 |
| Zona | WO2014/198223 |
| D2B | WO2009/130575 |

ELISA Data Human PSMA was coated on a 96 well plate at 3 μg/ml for at least two hours at room temperature. The plate was washed with wash buffer (0.05% Tween-20 in PBS), then blocked with blocking buffer (3% BSA in PBS) washed again using wash buffer. After which antibodies were titrated from 66.6 nM to 0.013 nM and added to the plate and incubated at room temperature for one hour, after which the plate was washed and anti-human-IgG-HRP added, the plate was incubated for a further 1 hour at room temperature, before being washed and TMB detection reagent added, the reaction was stopped by the addition of 1M HCl to the wells, after which the plate was read at 450 nm using the SpectraMax plate reader.

TABLE 3
Results

| PSMA Antibody | EC₅₀ (nM) |
|---|---|
| J415 | 24.84 |
| Mdx 2A10 | 0.1145 |
| PMF-A10 | 0.1615 |
| D2B | 0.173 |
| J591 | 0.2708 |
| ZONA | 0.3106 |

These results show that apart from the J415 antibody, all the tested antibodies bound with sub-nM EC₅₀, with 2A10 have the lowest (best) value.

Cytotoxicity Assessment on LNCaP Cells

Six different anti-PSMA antibodies were conjugated to the PBD SG3199 (drug-linker SG3249), essentially as described in Example 5.

Cells were diluted to 5×10⁴ cells/mL in complete growth medium with 100 μL of cells added to wells in an EDGE plate. The cells were incubated at 37° C./5% CO₂ for 2-6 hours to allow time for the cells to adhere. The ADCs were diluted in an 11 point, 1 in 4 serial dilutions, from 50 μg/mL to 47.7 pg/mL, leaving a final negative control sample i.e. no ADC, each concentration was run in duplicate. The diluted ADCs were added to the EDGE plate containing the target cells, and the plate was incubated in a humidified incubator for 5 days (˜120 hours). To determine the cytotoxic effect of the ADCs, Cell Titre Glow (Promega) was used, 40 μL of the read solution was added to each well on the plate and incubated at 37° C./5% CO₂ for 1-5 hours. After the incubation, the plate was read on an optical reader (Molecular Devices Spectramax i3X) and the data analysed by the software inherent to the machine (Softmax Pro). Overall the data is a result of running each cell line in triplicate.

TABLE 4
Results

| Construct | EC₅₀ (pM) |
|---|---|
| B12-SG3249 | 13988 |
| D2B-SG3249 | 35.58 |
| J415-SG3249 | 119.7 |
| J591-SG3249 | 516.9 |
| MDX 2A10-SG3249 | 110.5 |
| PMF-A10-SG3249 | 8047 |
| Zona-SG3249 | 75.43 |

### Example 3— Pharmacokinetic Assessment of Half-Life of PSMA Abs in the Humanized FcRn Model

Materials and Methods

The pharmacokinetic parameters of 6 PSMA-specific monoclonal antibodies (2A10, 4A3 (Medarex, WO2003/064606), D2B, J415, J591 and J591 formatted as a DVD-Ig) were determined following IV administration to human neonatal Fc receptor (FcRn) transgenic mice on a severe combined immunodeficient (SCID) background (Tg32 SCID mice, Jackson Laboratories). Animals (n=5/group) received a single IV injection of each antibody at 10 mg/kg. Blood samples were then taken through the retro-orbital sinus at 30 min, 6 h, and 1, 3, 5, 7, 10, 14, 17, 21 and 28 days post injection and processed to plasma. Circulating antibodies concentrations were then determined using a human IgG-specific enzyme-linked immunosorbent assay (ELISA). The data was then plotted analysed using Microsoft Excel to determine PK parameters, including half-life, clearance, area under the curve (AUC) then plotted and volume of distribution.

Results

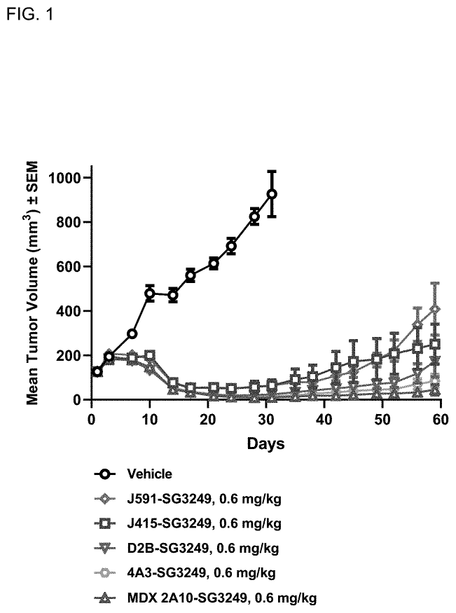

The exposure profiles following single dose administration of 6 PSMA-specific monoclonal antibodies are shown in FIG. 1. Antibodies 2A10, 4A3, D2B and J415 all exhibited significantly better exposure profiles than J591 and J591 DVD-Ig, which both showed evidence of a more rapid decline in exposure. This was reflected in the pharmacokinetic parameters, with J591 and J591 DVD-Ig showing a similar mean half life (6.3 and 6.6 days, respectively), and 2A10, 4A3, D2B and J415 exhibiting half lives of 15.2, 13.1, 13.6 and 10.8 days, respectively (Table 5).

TABLE 5
Half-Life, Clearance and AUC data
for different anti-PSMA antibodies

| | | | | Volume of |
|---|---|---|---|---|
| | Half-Life | Clearance | AUC | Distribution |
| | Days | ml/Days | μg-days/ml | Days |
| 2A10 | 15.2 | 0.286 | 807 | 6.46 |
| sem | 0.9 | 0.122 | 192 | 2.95 |
| 4A3 | 13.1 | 0.310 | 973 | 6.18 |
| sem | 1.2 | 0.118 | 348 | 2.67 |
| D2B | 13.6 | 0.158 | 1194 | 2.86 |
| sem | 2.8 | 0.028 | 254 | 0.44 |
| J415 HAKA | 10.8 | 0.167 | 1170 | 2.58 |
| sem | 1.2 | 0.007 | 18 | 0.25 |
| J591 | 6.3 | 0.485 | 566 | 4.08 |
| sem | 0.8 | 0.102 | 125 | 0.55 |
| J591 DVD IgG | 6.6 | 0.233 | 943 | 2.20 |
| sem | 0.6 | 0.014 | 57 | 0.12 |

### Example 4—In Vivo Efficacy of Different Anti-PSMA Antibodies Conjugated to a PBD in the CWR22Rv1 Xenograft Model

Materials and Methods

Five different anti-PSMA antibodies were conjugated to the PBD SG3199 (drug-linker SG3249), essentially as described in Example 5.

Male severe combined immunodeficient mice (Fox Chase SCID®, CB17/lcr-Prkdcscid/IcrlcoCrl, Charles River) were nine weeks old with a body weight (BW) range of 17.1 to 22.5 g on Day 1 of the study. Each mouse was injected subcutaneously (s.c.) in the right flank with 1×10⁷ 22 Rv1 cells in 50% matrigel. Tumor were measured in two dimensions using calipers, and volume was calculated using the formula:

Tumor Volume (mm³)=w2×l/2

where w=width and l=length, in mm, of the tumor. Tumor weight may be estimated with the assumption that 1 mg is equivalent to 1 mm³ of tumor volume.

Nineteen days later, designated as Day 1 of the study, mice were sorted into treatment groups (n=10 per group) with individual tumor volumes ranging from 108 to 144 mm³ and group mean tumor volumes of 126 mm³. On Day 1 of the study, drugs were administered intravenously (i.v.) in a single injection (qd×1) via tail vein injection. The dosing volume was 0.2 mL per 20 grams of body weight (10 mL/kg), and was scaled to the body weight of each individual animal. Tumors were measured using calipers twice per week, and each animal was euthanized when its tumor reached the endpoint volume of 1000 mm³ or at the end of the study (Day 59), whichever came first.

Results

TABLE 6
In vivo anti-tumour activity

| Response summary | PR | CR | TFS |
|---|---|---|---|
| Vehicle | 0 | 0 | 0 |
| J591-SG3249 (0.6 mg/kg) | 7 | 1 | 1 |
| J415-SG3249 (0.6 mg/kg) | 5 | 3 | 0 |
| D2B-SG3249 (0.6 mg/kg) | 5 | 5 | 1 |
| 4A3-SG3249 (0.6 mg/kg) | 4 | 6 | 2 |
| MDX 2A10-SG3249 (0.6 mg/kg) | 2 | 8 | 6 |

PR = partial responder.
CR = complete responder.
TFS = tumour-free survivor

In a PR response, the tumor volume was 50% or less of its Day 1 volume for three consecutive measurements during the course of the study, and equal to or greater than 13.5 mm³ for one or more of these three measurements.

In a CR response, the tumor volume was less than 13.5 mm³ for three consecutive measurements during the course of the study.

An animal with a CR response at the termination of a study was additionally classified as a tumor-free survivor (TFS).

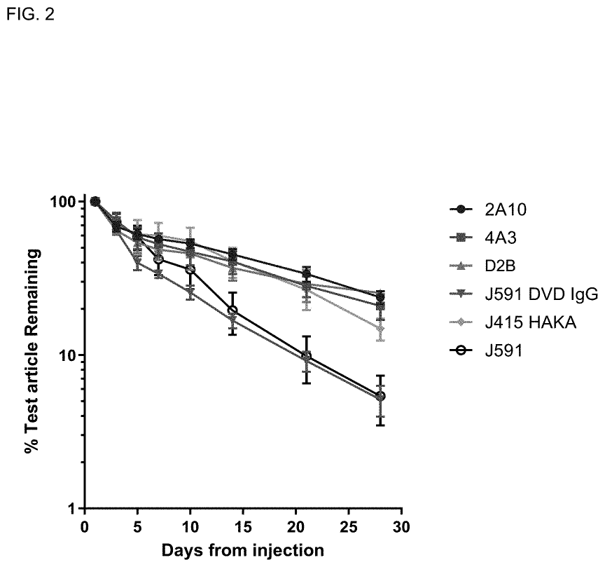

See also FIG. 2.

Conclusions

All ADCs show specific activity in the CWR22Rv1 model. 2A10-SG3249 was the best performing ADC whilst J591-SG3249 was the worst ADC, confirming the in vitro data in the previous examples.

### Example 5— Assessment of 2A10 PBD ADC Cytotoxicity Against Multiple Cell Lines

Based on the binding data, in vitro and in vivo cytotoxicity as well as PK analysis in humanized FcRn mice, antibody 2A10 was the best performing antibody. 2A10 was selected for further studies using different PBDs.

Materials and Methods

Synthesis of ADCs

Different PBD-based ADCs were constructed linked to antibody 2A10. Antibody B12, an human IgG1 non-binding antibody was used as an isotype control: it does not specifically bind PSMA.

2A10-B1 contains SG2000 as the PBD and the linker structure as shown above for compound B1. The synthesis of this drug linker is described above. The drug-linker was conjugated to the 2A10 antibody (previously treated with EndoS to trim back to GlcNAc at the N-linked glycosylation site at N-297) using the Synaffix GlycoConnect™ method (van Geel et al., 2015, Bioconjugate Chemistry 26 (11), 2233-2242).

2A10-B2 differs from 2A10-B1 in that the PBD is SG3249— see compound C2 above.

2A10-B3 is SG3249 (SG3199 as the PBD— see compound C3 above) conjugated to 2A10 via hinge region cysteine resides essentially as described in Zammarchi et al., 2016. Mol Cancer Ther 15: 2709 In brief, antibody was buffer exchanged into a histidine buffer at pH 6 using tangential flow filtration, pH was adjusted to 7.5 using a TRIS/EDTA pH 8.5 buffer, and the solution was reduced with Tris (2-carboxyethyl) phosphine reductant. Dimethylacetamide and SG3249 (threefold excess relative to antibody) were added to the solution. The conjugation reaction was incubated, then quenched with threefold molar excess of N-acetyl cysteine and incubated again. The pH was then decreased to 6.0 using histidine hydrochloride solution and the generated 2A10-SG3249 was purified by tangential flow filtration, filtered, and stored at −70° C. Final yield was estimated by ultraviolet-visible spectrophotometry based on starting antibody.

MAIA 2A10-B4 contains compound C4 as the drug-linker (see WO2018/069490 for synthesis). The heavy chain of 2A10 has been engineered to introduce a cysteine residue at position 242 of SEQ ID NO: 2 (between S and V). Compound C4 is conjugated at this introduced cysteine. For conjugation, the antibody was reduced using 40 molar equivalents of DTT, incubated over night at room temperature. After incubation the DTT was removed via a G25 desalting column and equilibrated with PBS. The antibody was then reoxidised using 25 molar equivalents of dehydroascorbic acid, with incubation for 4 hours at room temperature after which 4 molar equivalents of SG3600 was added and then desalted into 30 mM Histidine, 175 mM Sucrose, pH6.0.

MAIA 2A10-B5 contains compound C5 as the drug-linker (see WO2019/034764 for synthesis). The heavy chain of 2A10 has been engineered to introduce a cysteine residue at position 242 of SEQ ID NO: 2 (between S and V). Compound C5 is conjugated at this introduced cysteine. Conjugation was performed as described for MAIA 2A10-B4.

ADC Cytoxicity Assay

As described in Example 2.

Results

The results in Table 7 showed that the ADCs based on 2A10 were much more potent than the B12 control antibody conjugates and that all the conjugates tested were highly potent in the 100's of pM range IC50's on LNCaP cells.

TABLE 7
Cytotoxicity results with ADCs - LNCaP Cells

| | Experiment 1 | | | | Experiment 2 | | | | Average |
|---|---|---|---|---|---|---|---|---|---|
| Description | 1 | 2 | 3 | 4 | 1 | 2 | 3 | 4 | IC50 (pM) |
| 2A10-B1 | 334.1 | | | | 494 | | | | 414.1 |
| B12-B1 | 2.53E⁺⁰⁵ | | | | 5.60E⁺⁰⁵ | | | | 406,500.0 |
| 2A10-B2 | | 324.4 | | | | 272.7 | | | 298.6 |
| (DAR1) | | | | | | | | | |
| 2A10-B2 | | 117.4 | | | | 152 | | | 134.7 |
| (DAR2) | | | | | | | | | |
| B12-B2 | | 5.39E⁺⁰⁴ | | | | 5.60E⁺⁰⁵ | | | 306,950.0 |
| 2A10-B3 | 72.88 | 68.1 | 96.3 | | 174.6 | 145.9 | 138.3 | | 116.0 |
| B12-B3 | 1.23E⁺⁰⁴ | | | | 2.52E⁺⁰⁴ | | | | 18750.0 |
| MAIA 2A10-B4 | | | 300.2 | | | | 215.9 | | 258.1 |
| B12-B4 | | | 4.27E⁺⁰⁴ | | | | 9.84E⁺⁰⁵ | | 513,350.0 |
| MAIA 2A10-B5 | | | | 85.16 | | | | 129.6 | 107.4 |
| MAIA B12-B5 | | | | 4.85E⁺⁰⁵ | | | | 6.53E⁺⁰⁴ | 275,150.0 |

A number of publications are cited above to more fully describe and disclose the disclosures and the state of the art to which inventions herein may pertain. The entirety of each of the references mentioned in this disclosure are hereby is incorporated by reference.

1. An antibody drug conjugate of formula Ab-L-D where Ab is an antibody that binds to prostate-specific membrane antigen (PSMA), conjugated to a DNA-binding cytotoxin comprising a pyrrolobenzodiazepine (PBD) dimer, wherein the antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;
and L-D is a drug linker of formula (III) 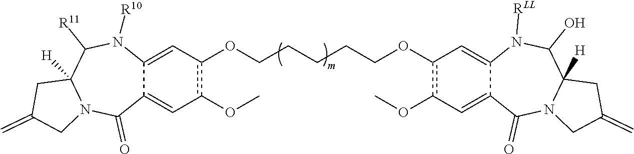
wherein:
(a) R^{LL} is a linker for connection to the antibody;
(b) (i) R¹⁰ and R¹¹ together form a double bond between the nitrogen and carbon atoms to which they are attached; (ii) R¹⁰ is a linker R^{LLA} for connection to the antibody and R¹¹ is OH; or (iii) R¹⁰ is a capping group R^{C} and R¹¹ is OH; and
(c) m is 0 or 1;
with the proviso that an antibody drug conjugate comprising either of the following drug linkers is specifically excluded:

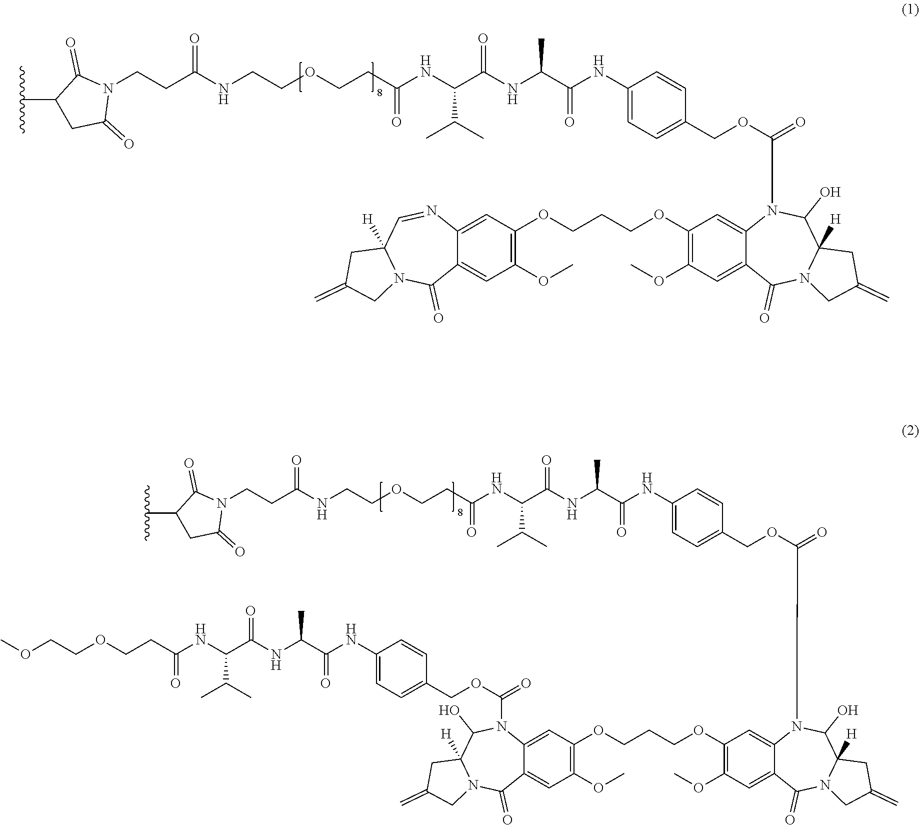

2. The conjugate according to claim 1 wherein the antibody comprises an immunoglobulin heavy chain variable region having the amino acid sequence shown in SEQ ID NO: 1.

3. The conjugate according to claim 1 wherein the antibody comprises an immunoglobulin light chain variable region having the amino acid sequence shown in SEQ ID NO: 2.

4. The conjugate according to claim 1 wherein m=0.

5. The conjugate according to claim 1 wherein R¹⁰ and R¹¹ together form a double bond between the nitrogen and carbon atoms to which they are attached.

6. The conjugate according to claim 1 wherein the conjugate comprises a linker of formula (IIa) between the cytotoxin and the antibody 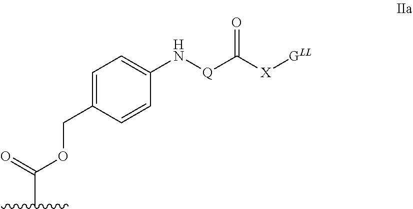
wherein:
Q is: 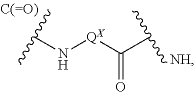
where Q^{X} is such that Q is an amino-acid residue, a dipeptide residue, a tripeptide residue or a tetrapeptide residue;
X, which is linked to G^{LL}, is: 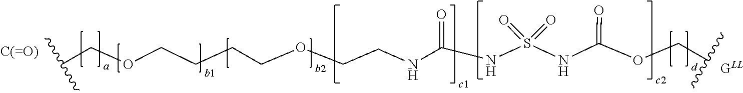
where a=0 to 5, b1=0 to 16, b2=0 to 16, c1=0 or 1, c2=0 or 1, d=0 to 5, wherein at least b1 or b2=0 and at least c1 or c2=0; and
G^{LL} is a linker group connected to the antibody.

7. The conjugate according to claim 1 wherein the conjugate comprises a cleavable linker between the cytotoxin and the antibody.

8. The conjugate according to claim 6 wherein c2=1 and b1 is from 2 to 8.

9. (canceled)

10. The conjugate according to claim 1 where the cytotoxin is conjugated to the antibody at an endogenous and/or engineered N-linked glycosylation site.

11. (canceled)

12. A method of treating an individual suffering from prostate cancer which method comprises administering to the patient an antibody drug conjugate according to claim 1.

13. The conjugate according to claim 1 wherein the drug linker (L-D) is:

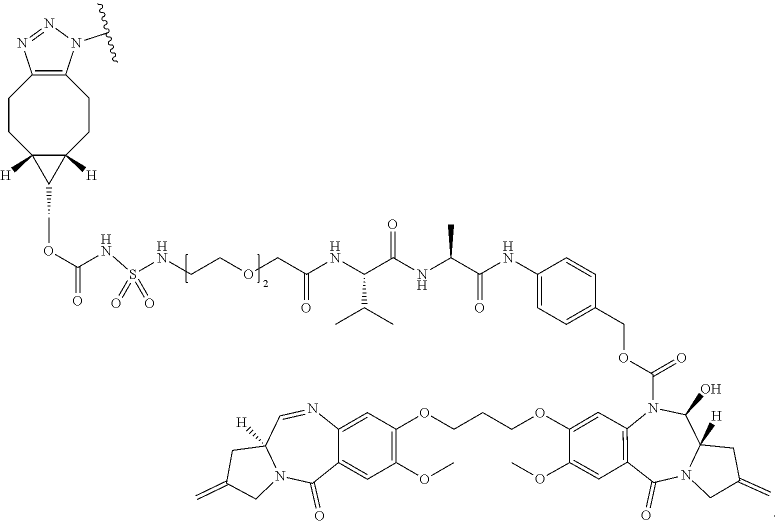

14. The conjugate according to claim 1 wherein the drug linker (L-D) is attached to the antibody (Ab) at an N-linked glycosylation site at Asn-297.

15. The conjugate according to claim 14 wherein the drug linker (L-D) is attached to the antibody (Ab) at an N-linked glycosylation site at Asn-297 through a trimmed Asn-GlcNAc residue.

16. The conjugate according to claim 15 wherein the N-linked glycosylation site is Asn-297-GlcNAc-GalNAc.

17. The conjugate according to claim 9 wherein the drug linker (L-D) is attached to the antibody (Ab) at an N-linked glycosylation site at Asn-297.

18. The conjugate according to claim 17 wherein the drug linker (L-D) is attached to the antibody (Ab) at an N-linked glycosylation site at Asn-297 through a trimmed Asn-GlcNAc residue.

19. The conjugate according to claim 18 wherein the N-linked glycosylation site is Asn-297-GlcNAc-GalNAc.

20. The conjugate according to claim 7 wherein the cleavable linker is a cathepsin cleavable sequence.

21. The conjugate according to claim 20, wherein the cathepsin cleavable sequence is Val-Ala or Val-Cit.


SEQUENCE LISTING (11 sequences)

SEQ ID NO: 1 - protein, 119 aa, synthetic construct
EVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSS

SEQ ID NO: 2 - protein, 107 aa, synthetic construct
AIQLTQSPSSLSASVGDRVTITCRASQDISSALAWYQQKPGKAPKLLIYDASSLESGVPSRFSGYGSGTDFTLTINSLQPEDFATYYCQQFNSYPLTFGGGTKVEIK

SEQ ID NO: 3 - protein, 5 aa, synthetic construct
SNWIG

SEQ ID NO: 4 - protein, 17 aa, synthetic construct
IIYPGDSDTRYSPSFQG

SEQ ID NO: 5 - protein, 10 aa, synthetic construct
QTGFLWSSDL

SEQ ID NO: 6 - protein, 11 aa, synthetic construct
RASQDISSALA

SEQ ID NO: 7 - protein, 7 aa, synthetic construct
DASSLES

SEQ ID NO: 8 - protein, 9 aa, synthetic construct
QQFNSYPLT

SEQ ID NO: 9 - protein, 450 aa, synthetic construct
EVQLVQSGAEVKKPGESLKISCKGSGYSFTSNWIGWVRQMPGKGLEWMGIIYPGDSDTRYSPSFQGQVTISADKSISTAYLQWSSLKASDTAMYYCARQTGFLWSSDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 4 aa, synthetic construct
GGFG

SEQ ID NO: 11 - protein, 4 aa, synthetic construct
GFGG